(19) United States
(12) Patent Application Publication
(10) Pub. No.: US 2012/0183751 A1
(43) Pub. Date: Jul. 19, 2012

(54) BASE GENERATOR, PHOTOSENSITIVE RESIN COMPOSITION, PATTERN FORMING MATERIAL COMPRISING THE PHOTOSENSITIVE RESIN COMPOSITION, PATTERN FORMING METHOD USING THE PHOTOSENSITIVE RESIN COMPOSITION AND PRODUCTS COMPRISING THE SAME

(75) Inventors: Mami Katayama, Tokyo-to (JP); Shunji Fukuda, Tokyo-to (JP); Katsuya Sakayori, Tokyo-to (JP)

(73) Assignee: DAI NIPPON PRINTING CO., LTD., Tokyo-to (JP)

(21) Appl. No.: 13/498,651

(22) PCT Filed: Sep. 29, 2010
(86) PCT No.: PCT/JP2010/066942

§ 371 Date: Mar. 28, 2012

(30) Foreign Application Priority Data
Sep. 30, 2009 (JP) 2009-226363

(51) Int. Cl. B32B 3/00; G03F 7/20; C07C 235/36; C09J 139/04; C08F 126/06; C08F 226/06; C09D 139/04; C09D 11/10; G03F 7/027; C07D 401/10

(52) U.S. Cl. 428/195.1; 430/283.1; 430/280.1; 430/325; 564/170; 546/189; 526/263; 524/548

(57) ABSTRACT

The present invention is to provide a photosensitive resin composition which has excellent resolution, is low in cost and is applicable to a wide range of applications due to the structure of a polymer precursor in which reaction into a final product is promoted by a basic substance or by heating in the presence of a basic substance. The present invention is also to provide a base generator which is applicable to such a photosensitive resin composition.

Disclosed is a base generator which has a specific structure and generates a base by exposure to electromagnetic radiation and heating. Also disclosed is a photosensitive resin composition which comprises the base generator and a polymer precursor in which reaction into a final product is promoted by a basic substance or by heating in the presence of a basic substance.

### TECHNICAL FIELD

The present invention relates to a base generator which generates a base by exposure to electromagnetic radiation and heating, and a photosensitive resin composition comprising the base generator. In particular, the present invention relates to the following: a photosensitive resin composition which can be suitably used as a material for products or components which are formed through a patterning process using electromagnetic radiation or through a curing acceleration process, a pattern forming material comprising the photosensitive resin composition, a pattern forming method, and an article comprising the resin composition.

### BACKGROUND ART

A photosensitive resin composition is used as a material for forming electronic components, optical products or optical elements, a material for forming layers, an adhesive, etc. Particularly, it is suitably used for products or components which are formed through a patterning process using electromagnetic radiation.

For example, polyimide, which is a polymer material, exhibits top-ranking performance in properties such as heat resistance, dimensional stability and insulation property among organic materials. Thus, it is widely used as an insulation material for electronic components, etc., and it is increasingly and actively used as a chip coating film of semiconductor elements, a substrate of flexible printed-wiring boards, and so on.

Also in recent years, to solve problems with polyimide, intensive investigations have been carried out into polybenzoxazole having a low water absorption property and a low dielectric constant, polybenzimidazole having excellent adhesion to substrates, and so on, which are processed in a similar manner to polyimide.

In general, polyimide shows poor solubility in solvents and is difficult to process. As the method for patterning polyimide in a desired shape, therefore, there is a method for obtaining a pattern of polyimide by patterning polyimide by exposure to light and development when it is in a state of polyimide precursor that has excellent solubility in solvents, and then imidizing the resultant by heating, etc.

Various methods are proposed for forming a pattern by using a polyimide precursor. Two typical examples are as follows:

(1) A method for forming a pattern by forming a resist layer comprising a photosensitive resin on a polyimide precursor which has no pattern forming ability

(2) A method for forming a pattern by introducing a photosensitive site to a polyimide precursor by a bond or coordination and forming a pattern by its action, or a method for forming a pattern by mixing a polyimide precursor with a photosensitive component to produce a resin composition and forming a pattern by the action of the photosensitive component

Typical patterning methods using method (2) include: (i) a method for obtaining a polyimide pattern in which a naphthoquinonediazide derivative which acts as a dissolution inhibitor before exposure to electromagnetic radiation and which produces a carboxylic acid and acts as a dissolution promoter after the exposure, is mixed with a polyimide precursor (polyamic acid) so that there is an increase in contrast between the dissolution rate of exposed regions in developers and that of unexposed regions in the same, thereby forming a pattern; thereafter, the pattern is imidized to obtain a polyimide pattern (patent literature 1) and (ii) a method for obtaining a polyimide pattern in which a methacryloyl group is introduced to a polyimide precursor via an ester bond or ionic bond; a photoradical generator is added thereto to crosslink exposed regions so that there is an increase in contrast between the dissolution rate of the exposed regions in developers and that of unexposed regions in the same, thereby forming a pattern; thereafter, the pattern is imidized to obtain a polyimide pattern (patent literature 2).

Compared with method (1), method (2) needs no resist layer, so that the process can significantly simplified. However, method (i) is problematic in that the original properties of polyimide cannot be obtained when the added amount of the naphthoquinonediazide derivative is increased for increasing the dissolution contrast. Method (ii) is problematic in that there is a limitation on the structure of the polyimide precursor.

There is a report of other patterning method (iii) which is a method for obtaining a polyimide pattern in which a polyimide precursor (polyamic acid) is mixed with a photobase generator; the mixture is exposed to light and then heated to promote cyclization by the action of bases generated by the exposure and thus to decrease the solubility of the polyimide precursor in developers so that there is an increase in contrast between the dissolution rate of exposed regions in developers and that of unexposed regions in the same, thereby forming a pattern; thereafter, the pattern is imidized to obtain a polyimide pattern (patent literature 3).

Other examples of the photosensitive resin composition comprising a photobase generator include a photosensitive resin composition comprising an epoxy compound (for example, patent literature 4). The photobase generator is exposed to light to generate amines in a layer that contains the epoxy compound, so that the amines act as an initiator or catalyst and cure the epoxy compound in exposed regions only, thereby forming a pattern.

Also, there is an example which uses a photosensitive resin composition comprising a base-reactive resin and a photocyclization-type photobase generator which generates an amine compound without involving decarboxylation reaction by exposure to light (patent literature 5). Since the photobase generator has excellent resistance to high temperatures, a pattern can be formed without generating a base at unexposed regions by heating.

### CITATION LIST

Patent literature 1: Japanese Patent Application Laid-Open (JP-A) No. S52-13315
Patent literature 2: JP-A No. S54-145794
Patent literature 3: JP-A No. H8-227154
Patent literature 4: JP-A No. 2003-212856
Patent literature 5: JP-A No. 2009-80452

### SUMMARY OF INVENTION

### Technical Problem

A photosensitive resin composition comprising a photobase generator can be produced by a simple process because a photosensitive polymer precursor can be obtained simply by mixing an existing polymer precursor with a photobase generator at a predetermined ratio. In particular, the photosensitive resin composition comprising the photobase generator provides the benefit of broad utility to polyimide precursors which conventionally have a limitation on the structure of usable precursor compounds because of applicability to polyimide precursors of various structures. However, as shown in the below-described comparative example, conventional photobase generators are problematic in that since they have low sensitivity, a large amount of exposure to electromagnetic radiation is needed. They are also problematic in that the large amount exposure to electromagnetic radiation leads to a decrease in throughput per unit time.

For example, in the case where a photobase generator is combined with a polyimide precursor, due to a mechanism in which only an exposed region is imidized and becomes insoluble in developers by the catalytic action of bases generated by exposure, if the polyimide precursor is a polyimide precursor that is originally highly soluble in developers, the exposed region also has a high dissolution rate so that there is a limitation in increasing the dissolution contrast between exposed and unexposed regions.

As the dissolution contrast between the exposed and unexposed regions becomes larger, the remaining thickness ratio of a pattern thus obtained after development becomes larger and the shape of the pattern becomes better. However, conventional photosensitive compositions necessitate controlling developer concentration or photobase generator usage and adding a dissolution promoter, resulting in a small process margin.

The present invention was achieved in light of these circumstances. A main object of the present invention is to provide a base generator which has excellent sensitivity and can be used in combination with any kind of polymer precursor, and a photosensitive resin composition which has excellent sensitivity, provides a large dissolution contrast between exposed and unexposed regions, and can form a well-shaped pattern with keeping a sufficient process margin.

### Solution to Problem

The base generator of the present invention comprises a compound having two or more partial structures each represented by the following general formula (1) per molecule and generates a base by exposure to electromagnetic radiation and heating:

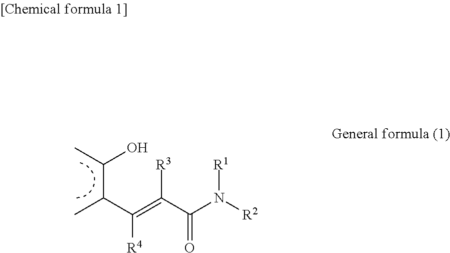

wherein R¹ and R² are each independently a hydrogen or an organic group and may be the same or different; at least one of R¹ and R² is an organic group; R¹ and R² may be bound to form a cyclic structure which may contain a heteroatom but does not contain an amide bond; R³ and R⁴ are each independently one selected from the group consisting of a hydrogen, a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group and an organic group and may be the same or different.

Due to having the partial structure represented by the chemical formula (1), the base generator having the above-specified structure generates a basic substance when it is subjected to a combination of exposure to electromagnetic radiation and heating, even at a small amount of exposure to electromagnetic radiation; therefore, the base generator is a base generator which has high sensitivity, can be used in combination with any kind of polymer precursor, and has broad utility.

The photosensitive resin composition of the present invention comprises a polymer precursor in which reaction into a final product is promoted by a basic substance or by heating in the presence of a basic substance, and the base generator of the present invention.

In the present invention, the base generator which comprises a compound having two or more partial structures each represented by the above chemical formula (1) per molecule and generates a base by exposure to electromagnetic radiation and heating, is combined with the polymer precursor in which reaction into a final product is promoted by a basic substance or by heating in the presence of a basic substance; therefore, the photosensitive resin composition of the present invention has excellent sensitivity, provides a large dissolution contrast between exposed and unexposed regions, and can form a well-shaped pattern with keeping a sufficient process margin.

In the present invention, the base generator is preferably a compound represented by the following general formula (2), a compound having a repeating unit represented by the following general formula (2′) or a compound represented by the following general formula (3)

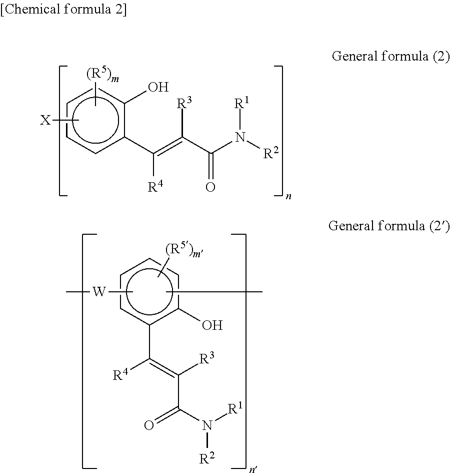

wherein R¹, R², R³ and R⁴ are the same as those of the general formula (1); n or n′ R¹s may be the same or different; n or n′ R²s may be the same or different; n or n′ R³s may be the same or different; n or n′ R⁴s may be the same or different; X is a direct bond or n-valent chemical structure to which two or n structures shown in the brackets are bound; W is a direct bond or a divalent linking group; n and n′ are each an integer of 2 or more; R⁵ and R^{5′} are each independently one selected from the group consisting of a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group, an amino group, an ammonio group and an organic group; m is 0 or an integer of 1 to 3; m′ is 0 or an integer of 1 or 2; two or more R⁵s may be the same or different and may be bound to form a cyclic structure which may contain a heteroatom; and two or more R^{5′}s may be the same or different and may be bound to form a cyclic structure which may contain a heteroatom;

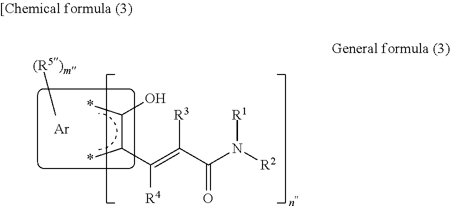

wherein R¹, R², R³ and R⁴ are the same as those of the general formula (1); n″ R¹s may be the same or different; n″ R²s may be the same or different; n″ R³s may be the same or different; n″ R⁴s may be the same or different; Ar is an aromatic hydrocarbon which has 6 to 24 carbon atoms and which may have a substituent, and which has n″ partial structures shown in the brackets; n″ is an integer of 2 or more; R^{5″} is one selected from the group consisting of a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group, an amino group, an ammonio group and an organic group; m″ is 0 or an integer of 1 or more; and two or more R^{5″}s may be the same or different and may be bound to form a cyclic structure which may contain a heteroatom.

In the present invention, the base generator preferably has a 5% weight loss temperature of 100° C. or more and 350° C. or less. When the 5% weight loss temperature is high, a coating film can be formed in a drying condition which minimizes the influences of a residual solvent. Therefore, it is possible to suppress a decrease in dissolution contrast between exposed and unexposed regions due to the influence of the residual solvent. On the other hand, when the 5% weight loss temperature is too high, base generator-derived impurities may remain in a final product and may deteriorate the properties of the product.

In the present invention, the base generator preferably has absorption at least one of electromagnetic wavelengths of 365 nm, 405 nm and 436 nm, from the point of view that the types of applicable polymer precursors are increased further.

In the photosensitive resin composition of the present invention, preferably used as the polymer precursor is one or more kinds selected from the group consisting of a compound having an epoxy group, isocyanate group, oxetane group or thiirane group, a polymer having an epoxy group, isocyanate group, oxetane group or thiirane group, a polysiloxane precursor, a polyimide precursor and a polybenzoxazole precursor.

In the photosensitive resin composition of the present invention, the polymer precursor is preferably soluble in basic solutions, from the point of view that a large dissolution contrast between the exposed and unexposed regions is obtained.

In an embodiment of the present invention, a polyimide precursor such as polyamic acid or a polybenzoxazole precursor can be used as the polymer precursor of the photosensitive resin composition. The use of such a polymer precursor provides a photosensitive resin composition with excellent physical properties such as heat resistance, dimensional stability and insulation property. The polyimide precursor is preferably a polyamic acid in terms of availability of raw materials.

The present invention also provides a photosensitive resin composition comprising a polymer having a repeating unit represented by the following general formula (2-4) as an essential component:

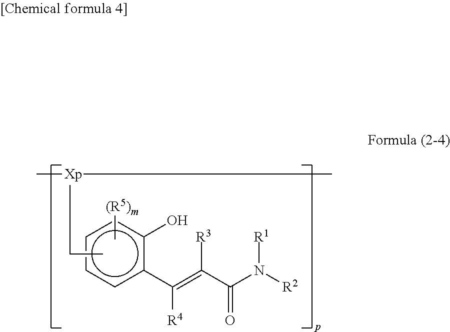

wherein R¹, R², R³, R⁴, R⁵ and m are the same as those of the general formula (2); Xp is a repeating unit of the polymer; and p is a number of 2 or more.

The present invention also provides a pattern forming material comprising the photosensitive resin composition of the present invention.

The present invention also provides a pattern forming method using the photosensitive resin composition.

The pattern forming method of the present invention is a method for forming a pattern by forming a coating film or molded body with the photosensitive resin composition, exposing the coating film or molded body to electromagnetic radiation in a predetermined pattern, heating the coating film or molded body after or at the same time as the exposure to change the solubility of the exposed region, and then developing the coating film or molded body.

In the pattern forming method, the polymer precursor is used in combination with the base generator which is a compound as represented by the above formula (1); therefore, it is possible to form a pattern by development without using a resist film which is for protecting the surface of the coating film or molded body comprising the photosensitive resin composition from developers.

The present invention also provides an article selected from a printed product, a paint, a sealing agent, an adhesive, a display device, a semiconductor device, an electronic component, a microelectromechanical system, a stereolithography product, an optical element or a building material, wherein at least part of each of which articles comprises the photosensitive resin composition or a cured product thereof.

### Advantageous Effects of Invention

Because of having the partial structure represented by the formula (1), the base generator of the present invention generates a base by exposure to electromagnetic radiation and the base generation is promoted by heating. Especially because the base generator of the present invention has a specific structure of having two or more partial structures each represented by the formula (1) per molecule, the base generator has greater sensitivity than conventionally-used photobase generators. When used for a photosensitive resin composition, the base generator of the present invention can be used in combination with any kind of polymer precursor.

The photosensitive resin composition of the present invention is a highly sensitive photosensitive resin composition because the base generator of the present invention contained has greater sensitivity than conventionally-used photobase generators. When the photosensitive resin composition of the present invention is subjected to exposure to electromagnetic radiation and heating, the solubility of the polymer precursor is changed by a base which is derived from the base generator; moreover, when the base is generated, the base generator loses the phenolic hydroxyl group and thus changes its solubility in basic aqueous solutions. Therefore, it is possible to further increase the difference between the solubility of the exposed region and that of the unexposed region. As a result of obtaining a large dissolution contrast between the exposed and unexposed regions, it is possible to obtain a well-shaped pattern with keeping a sufficient process margin.

Also in the photosensitive resin composition of the present invention, unlike acid, the base causes no metal corrosion; therefore, the photosensitive resin composition can form a more highly reliable cured film.

When a heating process is included in a pattern forming process, the photosensitive resin composition of the present invention can utilize the heating process as a heating for promoting base generation and thus is advantageous in that the amount of exposure to electromagnetic radiation can be decreased by the utilization of the heating process. Therefore, compared with conventional resin compositions which produce a base only by exposure to electromagnetic radiation, the photosensitive resin composition of the present invention can realize process rationalization when it is used in a process that includes such a heating process.

### DESCRIPTION OF EMBODIMENTS

Hereinafter, the present invention will be described in detail.

In the present invention, (meth)acryloyl means acryloyl and/or methacryloyl. (Meth)acryl means acryl and/or methacryl. (Meth)acrylate means acrylate and/or methacrylate.

Also in the present invention, except when a specific wavelength is mentioned, electromagnetic radiation encompasses not only electromagnetic waves having wavelengths in the visible and non-visible regions but also particle beams such as an electron beam, and radiation or ionizing radiation, each of which is a collective term that includes electromagnetic waves and particle beams. In this description, exposure to electromagnetic radiation is also referred to as exposure to light. Electromagnetic waves having a wavelength of 365 nm, 405 nm and 436 nm may be referred to as i-line, h-line and g-line, respectively.

### <Base Generator>

The base generator of the present invention comprises a compound having two or more partial structures each represented by the following general formula (1) per molecule and generates a base by exposure to electromagnetic radiation and heating:

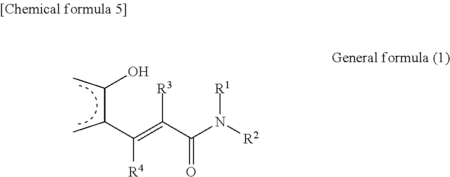

wherein R¹ and R² are each independently a hydrogen or an organic group and may be the same or different; at least one of R¹ and R² is an organic group; R¹ and R² may be bound to form a cyclic structure which may contain a heteroatom but does not contain an amide bond; R³ and R⁴ are each independently one selected from the group consisting of a hydrogen, a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group and an organic group and may be the same or different.

The base generator of the present invention is a kind of photobase generator. It generates a base only by exposure to electromagnetic radiation but the base generation is promoted by heating appropriately. The base generator of the present invention can generate a base efficiently by a combination of exposure to electromagnetic radiation and heating, with even a small amount of exposure to electromagnetic radiation. Therefore, it has higher sensitivity than conventional, so-called photobase generators. A photobase generator is an agent which is not active in a normal condition of ordinary temperature and pressure but generates a base when it is subjected to exposure to electromagnetic radiation as an external stimulus.

The base generator of the present invention has the above-specified structure; therefore, when it is exposed to electromagnetic radiation, as shown by the following formula, (—CR⁴═CR³—C(═O)—) in the formula (1) is isomerized into a cis isomer. Moreover, the cis isomer is cyclized by heating to generate a base (NHR¹R²). By the catalytic action of the base thus generated, it is possible to decrease reaction initiation temperature at which a reaction of a polymer precursor into a final product is initiated, or it is possible to initiate a curing reaction of a polymer precursor into a final product.

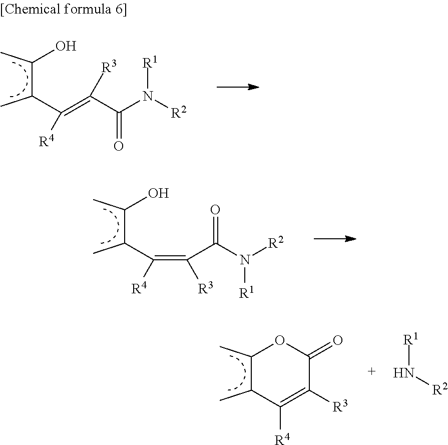

When the base generator of the present invention is cyclized, it loses the phenolic hydroxyl group to change the solubility thereof, thus having low solubility in a basic aqueous solution or the like. Because of this, when a polymer precursor contained in the photosensitive resin composition of the present invention is a polyimide precursor or polybenzoxazole precursor, the base generator has a function to further support the solubility decrease due to the reaction of the precursor into a final product, thereby making it possible to increase the dissolution contrast between exposed and unexposed regions.

Especially in the general formula (1), R¹ and R² contain no amide bond, and the base generator of the present invention comprises a compound having two or more partial structures each represented by the general formula (1) per molecule. In the present invention, therefore, the number of bases (NR¹R²) that can be generated per molecule is the same as the number of partial structures each represented by the general formula (1) and contained per molecule. That is, the base generator of the present invention differs from the structure as described in paragraph [0028] of patent literature 5, which is a structure in which two residues are bound to one diamine, each of the residues being the rest of the general formula (1) excluding NR¹R².

The base generator of the present invention comprises a compound which contains one or two or more partial structures each represented by the general formula (1) per aromatic hydrocarbon which functions as a light-absorbing group. Even in the case where one partial structure represented by the general formula (1) is contained per aromatic hydrocarbon which functions as a light-absorbing group, the base generator of the present invention has two or more partial structures each represented by the general formula (1) per molecule; therefore, the aromatic hydrocarbons, each of which functions as a light-absorbing group, have a structure that they are bound one another by a direct bond or linking group. In this case, since the binding to another aromatic hydrocarbon brings a substituent effect, each of the aromatic hydrocarbons receives influences such that the absorption wavelength is shifted to a longer wavelength side. Therefore, compared with the case where an unsubstituted benzene ring has one partial structure represented by the general formula (1), the base generator of the present invention has a further increase in sensitivity. On the other hand, in the case where two or more partial structures each represented by the general formula (1) is contained per aromatic hydrocarbon which functions as a light-absorbing group, since the other partial structure brings a substituent effect, one of the partial structures each represented by the general formula (1) receives influences such that the absorption wavelength is shifted to a longer wavelength side. Therefore, compared with the case where an unsubstituted benzene ring has one partial structure represented by the general formula (1), the base generator of the present invention has a further increase in sensitivity.

In the case where one partial structure represented by the general formula (1) is contained per aromatic hydrocarbon which functions as a light-absorbing group and the aromatic hydrocarbons have a structure that they are bound by a certain chemical structure, the solubility of the base generator of the present invention in organic solvents and the affinity of the same for a polymer precursor to be combined or the like can be increased by the selection of the chemical structure which functions as a linking group.

In the case where two or more partial structures each represented by the general formula (1) are contained per aromatic hydrocarbon which functions as a light-absorbing group, compared with a compound which has one base generating site per light-absorbing group, the base generator of the present invention is advantageous in that it can generate more bases in the same added amount.

Compared with the structure as described in paragraph [0028] of patent literature 5, which is a structure in which two light-absorbing groups are bound to one diamine, the base generator of the present invention shows higher base generation efficiency.

In the case where the base generator of the present invention comprises one partial structure represented by the general formula (1) per aromatic hydrocarbon which functions as a light-absorbing group, a compound represented by the following general formula (2) or a compound having a repeating unit represented by the following general formula (2′) is preferable from the point of view that it is easy to control the solubility and so on of the base generator by the selection of the structure of X or W and it is relatively easy to obtain such a compound.

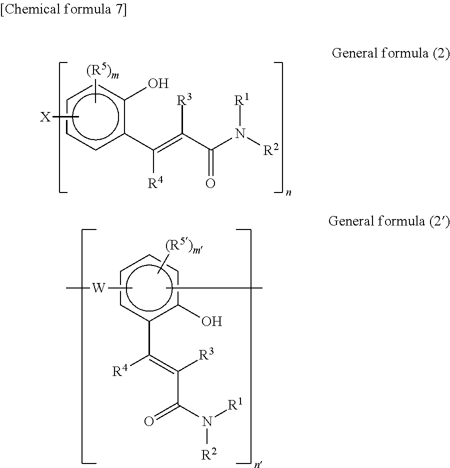

wherein R¹, R², R³ and R⁴ are the same as those of the general formula (1); n or n′ R¹s may be the same or different; n or n′ R²s may be the same or different; n or n′ R³s may be the same or different; n or n′ R⁴s may be the same or different; X is a direct bond or n-valent chemical structure to which two or n structures shown in the brackets are bound; W is a direct bond or a divalent linking group; n and n′ are each an integer of 2 or more; R⁵ and R^{5′} are each independently one selected from the group consisting of a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group, an amino group, an ammonio group and an organic group; m is 0 or an integer of 1 to 3; m′ is 0 or an integer of 1 or 2; two or more R⁵s may be the same or different and may be bound to form a cyclic structure which may contain a heteroatom; and two or more R^{3′}s may be the same or different and may be bound to form a cyclic structure which may contain a heteroatom.

In the general formula (2), in the case where chemical structure X which functions as a linking group is a direct bond, two structures, each of which is the structure that is shown in the brackets of the general formula (2), are directly bound to each other. In the present invention, “direct bond” means that no atom is present in between. In the case where chemical structure X is an n-valent structure, n structures, each of which is the structure that is shown in the brackets of the general formula (2), are bound to the n-valent structure.

In the present invention, the valence of chemical structure X does not contain the valence in the case where X has a substituent.

As the general formula (2), for example, there may be mentioned compounds having the following structures:

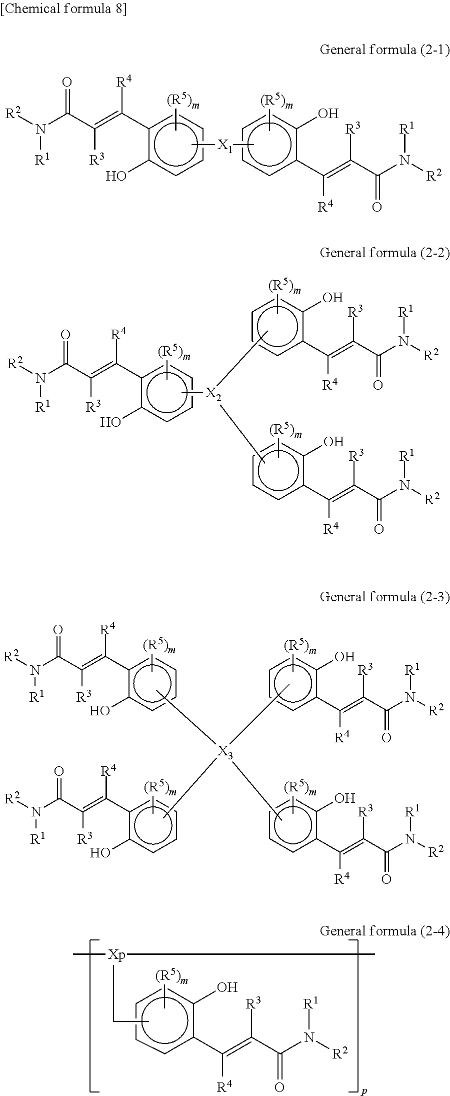

In the general formulae (2-1) to (2-4), R¹, R², R³, R⁴, R⁵ and m are the same as those of the general formula (2); X₁ is a divalent chemical structure; X₂ is a trivalent chemical structure; X₃ is a tetravalent chemical structure; Xp is a repeating unit of a polymer; p is a number of two or more.

In the general formula (2), chemical structure X may be a structure which has any chemical structure that is divalent or more. For example, there may be mentioned an organic group, an ether bond, a thioether bond, a carbonyl bond, a thiocarbonyl bond, an ester bond, an amide bond, an urethane bond, an imino bond (such as —N═C(—R)— or —C(═NR)— wherein R is a hydrogen atom or a monovalent organic group), a carbonate bond, a sulfonyl bond, a sulfinyl bond, an azo bond, a carbodiimide bond, etc. As the organic group, for example, there may be mentioned a linear, branched and/or cyclic saturated or unsaturated aliphatic hydrocarbon group, an aromatic hydrocarbon group and combinations thereof. Each of these examples may have inside thereof one or more bonds selected from the group consisting of an ether bond, a thioether bond, a carbonyl bond, a thiocarbonyl bond, an ester bond, an amide bond, an urethane bond, an imino bond (such as —N═C(—R)— or —C(═NR)— wherein R is a hydrogen atom or a monovalent organic group), a carbonate bond, a sulfonyl bond, a sulfinyl bond, an azo bond, a carbodiimide bond, etc. Examples of the divalent or more chemical structure other than organic group include siloxane, silane and borazine.

From the viewpoint of availability and ease of synthesis, a structure selected from the group consisting of an organic group, an ether bond, a thioether bond, a carbonyl bond, a thiocarbonyl bond, an ester bond and an amide bond is preferred as chemical structure X. As the organic group, from the viewpoint of cost, availability, ease of synthesis, solubility and heat resistance, an alkylene group, an alkenylene group, an alkynylene group are preferable. Moreover, one which contains inside thereof an ester bond, an ether bond, an amide bond or an urethane bond is also preferable.

In the compound represented by the general formula (2), as exemplified below, two or more partial structures bound to chemical structure X, each of which is the structure that is shown in the brackets, may be the same or different from one another in the substitution positions of the phenolic hydroxyl group and Z shown in the following structure. However, as shown in the general formula (1), a phenolic hydroxyl group and Z shown in the following structures are needed to be adjacent to each other.

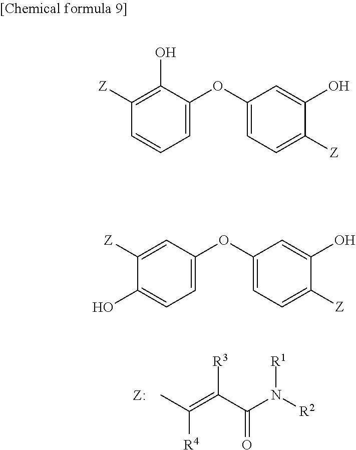

In the formulae, R¹, R², R³ and R⁴ are the same as those of the general formula (1).

Specific structures of the compound represented by the general formula (2) are exemplified below. However, the compound is not limited to the following examples.

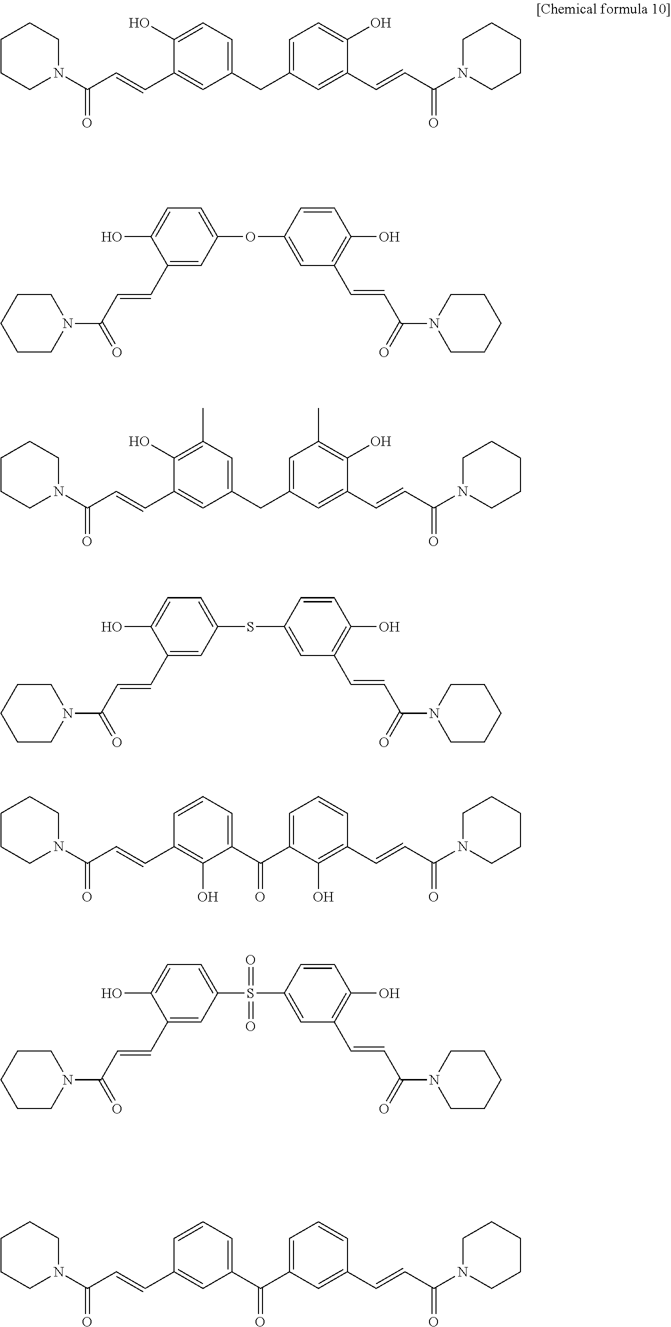

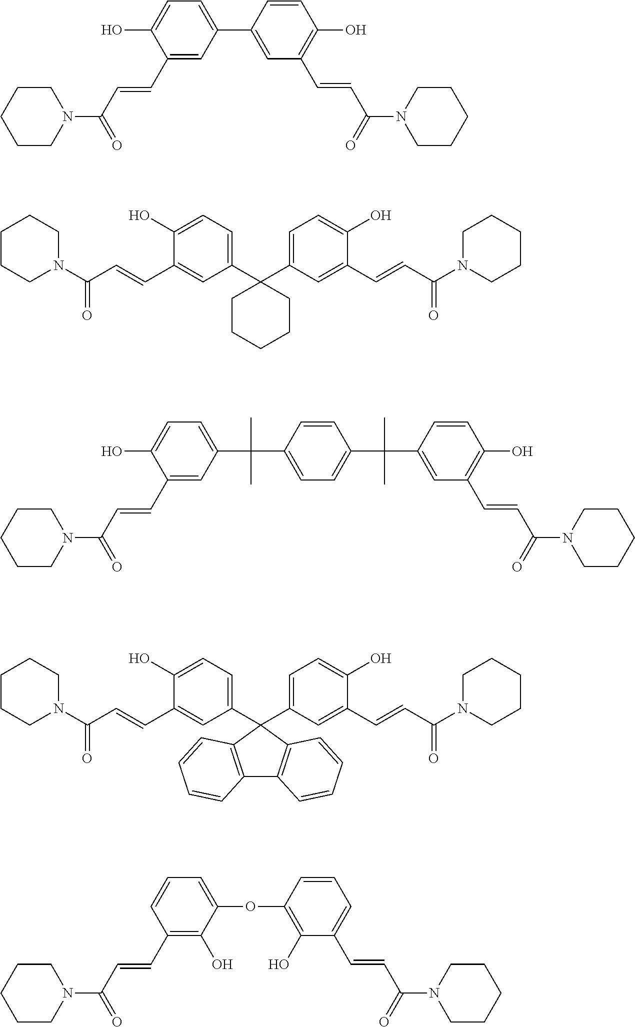

Like the polymer containing the repeating unit represented by the general formula (2-4), the compound represented by the general formula (2) may have a structure in which the polymer skeleton has plurality of structures in pendant form, each of which is the structure shown in the brackets of general formula (2). In this case, in the compound represented by the general formula (2), a repeating unit other than the repeating unit represented by the general formula (2-4) may be contained, and the repeating unit other than the repeating unit represented by the general formula (2-4) and plurality of Xp (in number of p) constitute n-valent chemical structure X. Also, Xp in number of p may be only one kind, two or more kinds or different. Specific examples of Xp include a structure derived from a monomer having an ethylenically unsaturated bond such as a (meth)acryloyl bond, and a structure containing an ester bond, an ether bond, an amide bond or an urethane bond. It is possible to appropriately select an optimal value to determine how much amount of the structures each of which is the structure shown in the brackets of general formula (2) should be contained in the polymer, considering materials to be used in combination, physical properties of a coating film which is the final product, etc.

Specific structures of the case where plurality of structures, each of which is the structure shown in the brackets of general formula (2), are contained in the polymer structure in pendant form, are exemplified below.

However, the case is not limited to these examples.

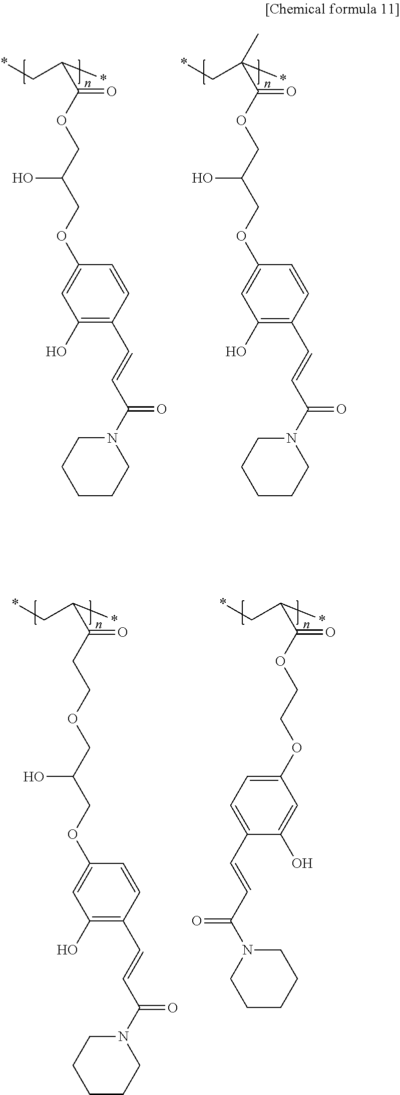

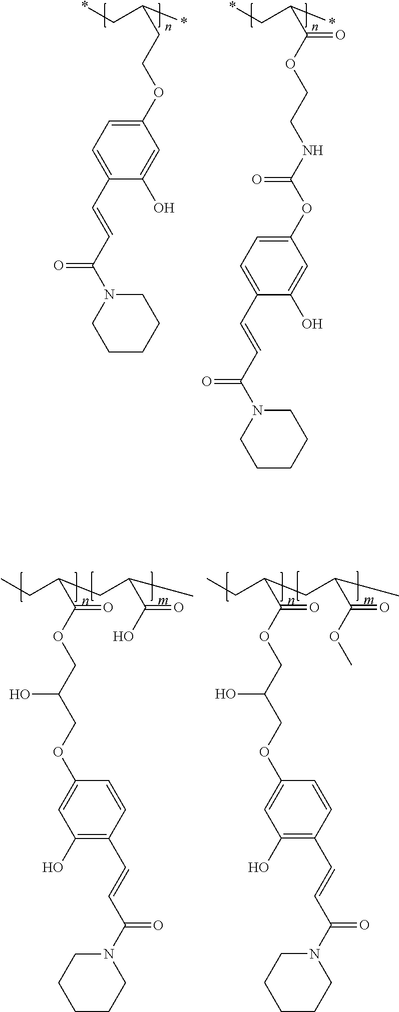

As the compound having a repeating unit represented by the general formula (2′), there may be mentioned a linear structure in which the repeating unit represented by the general formula (2′) has terminals. In the case where the repeating unit represented by the general formula (2′) has a linear structure, the terminal is not particularly limited as long as the effects of the present invention are not deteriorated. In the case where the compound having a repeating unit represented by the general formula (2′) has a linear structure, for example, there may be mentioned a compound represented by the following general formula (2′-1).

The compound having a repeating unit represented by the general formula (2′) may be a cyclic compound in which repeating units represented by the general formula (2′) are bound to form a cycle, like the compound represented by the following general formula (2′-2).

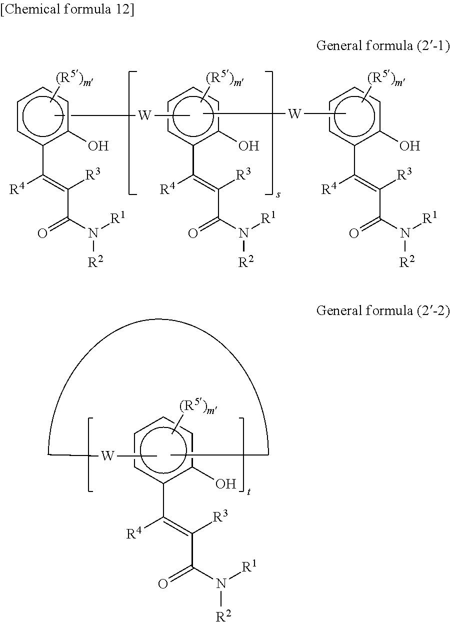

In the general formulae (2′-1) and (2′-2), R¹, R², R³, R⁴, R^{5′} and m′ are the same as those of the general formula (2′); s is an integer of 1 or more; and t is an integer of 3 or more.

In the compound having a repeating unit represented by the general formula (2′), W is a direct bond or a divalent linking group. As divalent linking group W, those that are the same as the divalent structures mentioned above in connection with chemical structure X can be used. Among them, from the viewpoint of availability and ease of synthesis, a structure selected from the group consisting of an organic group, an ether bond, a thioether bond, a carbonyl bond, a thiocarbonyl bond, an ester bond and an amide bond is preferred as divalent linking group W. As the organic group, from the viewpoint of cost, availability, ease of synthesis, solubility and heat resistance, an alkylene group, an alkenylene group, an alkynylene group are preferable. Moreover, one which contains inside thereof an ester bond, an ether bond, an amide bond or an urethane bond is also preferable.

As the general formula (2′), for example, there may be mentioned compounds having the following structures:

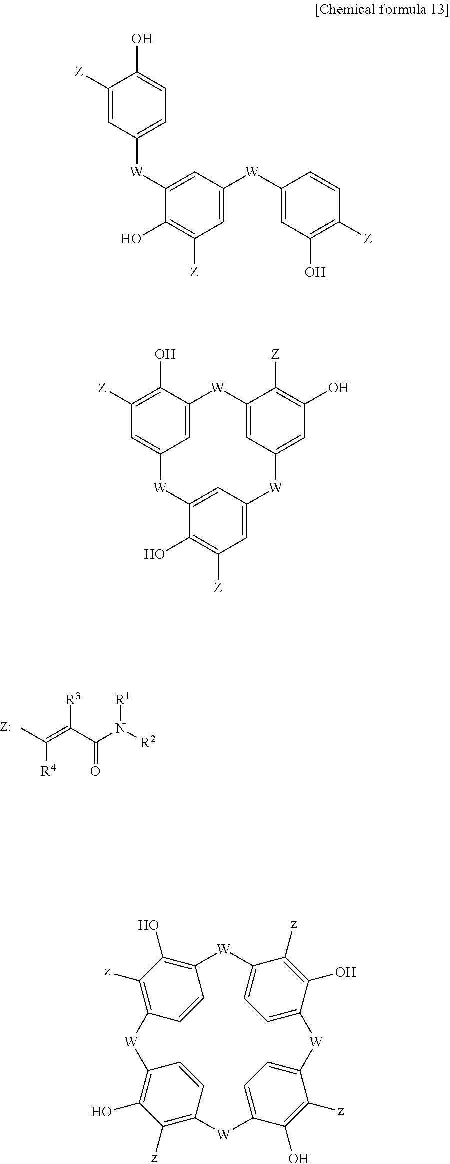

Specific structures of the compound represented by the general formula (2′) are exemplified below. However, the compound is not limited to the following examples.

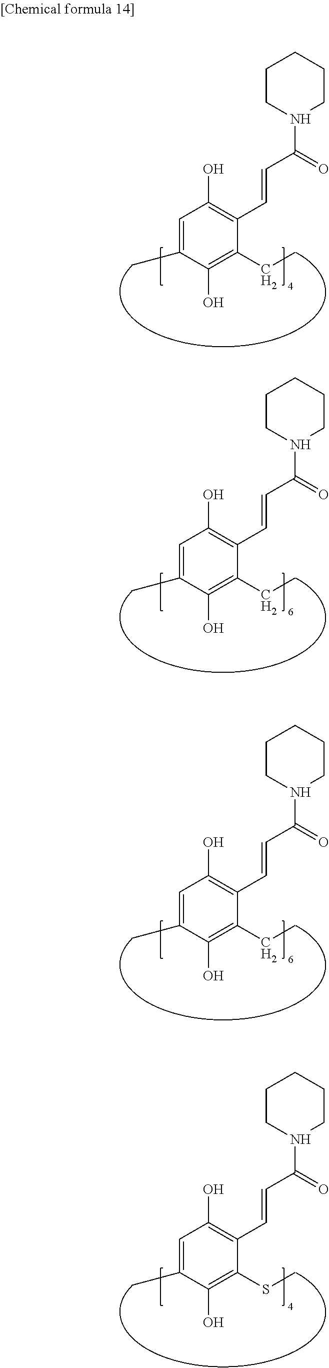

In the case where two or more partial structures each represented by the general formula (1) is contained per aromatic hydrocarbon which functions as a light-absorbing group, there may be mentioned a compound represented by the following general formula (3) as an example of the case:

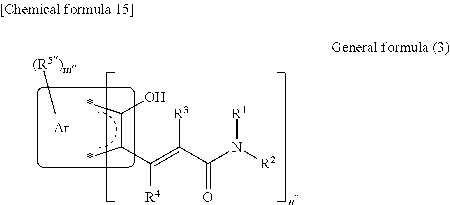

wherein R¹, R², R³ and R⁴ are the same as those of the general formula (1); n″ R¹s may be the same or different; n″ R²s may be the same or different; n″ R³s may be the same or different; n″ R⁴s may be the same or different; Ar is an aromatic hydrocarbon which has 6 to 24 carbon atoms and which may have a substituent, and which has n″ partial structures shown in the brackets; n″ is an integer of 2 or more; R^{5″} is one selected from the group consisting of a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group, an amino group, an ammonio group and an organic group; m″ is 0 or an integer of 1 or more; and two or more R^{5″}s may be the same or different and may be bound to form a cyclic structure which may contain a heteroatom.

In the general formula (3), Ar is an aromatic hydrocarbon which has 6 to 24 carbon atoms and which may have a substituent. For example, there may be mentioned benzene, naphthalene, fluorene, phenanthrene, anthracene and pyrene, each of which may have a substituent.

In the general formula (3), two cyclic conjugated carbon atoms in the brackets are bound to cyclic conjugated carbon atoms contained in aromatic hydrocarbon Ar at two positions each represented by * to constitute the aromatic hydrocarbon.

Examples of the compound represented by the general formula (3) include compounds having the following structures. In the compound represented by the general formula (3), as exemplified below, the two or more partial structures in the brackets may be adjacent to each other or phenolic hydroxyl groups may be adjacent to each other. However, as shown in the general formula (1), a phenolic hydroxyl group and Z shown in the following structures are needed to be adjacent to each other.

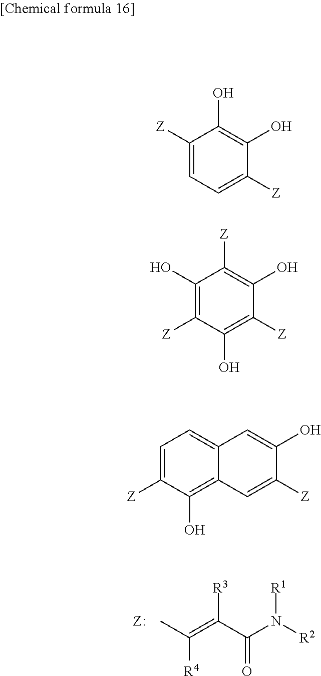

In the formulae, R¹, R², R³ and R⁴ are the same as those of the general formula (1).

Specific structures of the compound represented by the general formula (3) are exemplified below. However, the compound is not limited to the following examples.

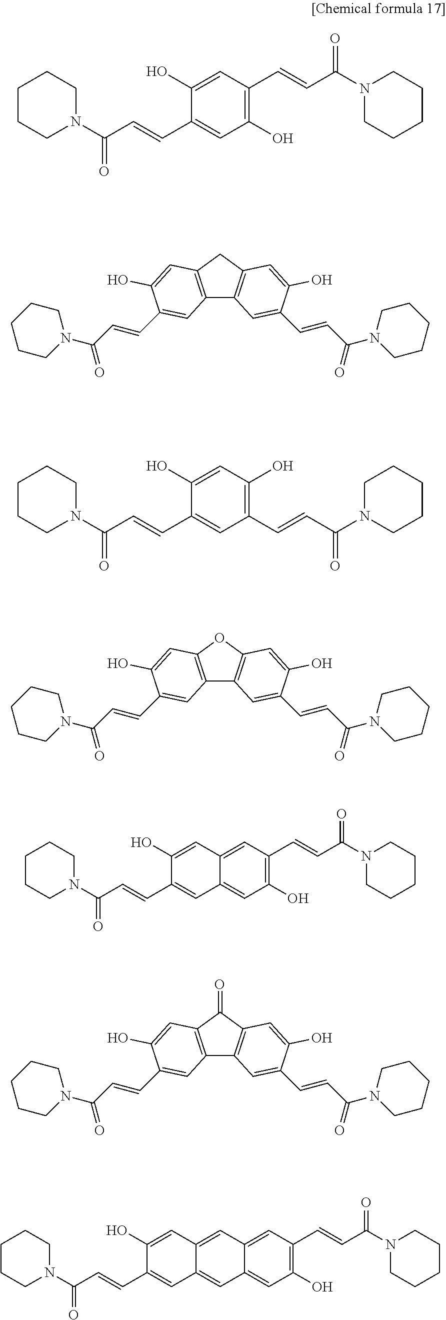

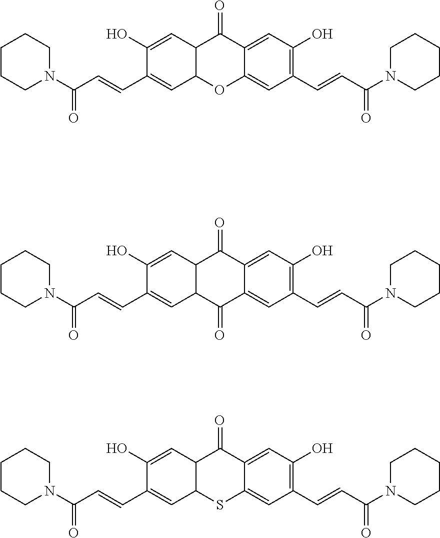

The compound having two or more partial structures each represented by the general formula (1) per molecule includes a structure which does not correspond to the general formula (2), (2′) or (3) such as a structure in which the structure represented by the general formula (3) is used as at least a part of the formula shown in the brackets of the general formula (2), as exemplified by the following formulae (a) and (b).

In the case where substituents R⁵s are bound to form a cyclic structure, examples of the general formula (2) include one in which other aryl group is used in place of the benzene ring in the brackets of the general formula (2), as exemplified by the following formula (c).

The compound having two or more partial structures each represented by the general formula (1) per molecule also includes a structure in which the partial structure represented by the general formula (1) is contained in chemical structure X of the general formula (2), as exemplified by the following formula (d) or (e). A structure like a dendrimer may be formed, such as a compound represented by the following formula (e).

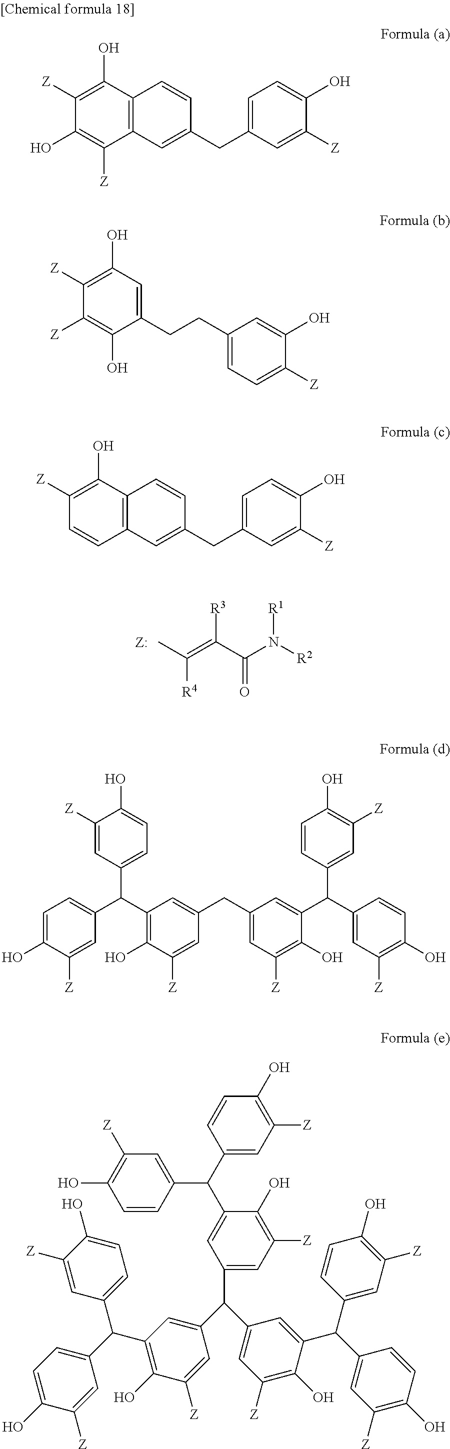

In the formula Z, R¹, R², R³ and R⁴ are the same as those of the general formula (1).

In the general formulae (1), (1′), (2), (2′) and (3), R¹ and R² are each independently a hydrogen or an organic group, and at least one of R¹ and R² is an organic group. Each of R¹ and R² contains no amide bond. That is, the thus-generated NHR¹R² is a base (in the present invention, “basic substance” is simply referred to as base) which has one NH group that can form an amide bond.

The base generator of the present invention generates no polyvalent base such as diamine and generates a monovalent base such as monoamine.

In the case where the thus-generated base is a polyvalent base having two or more NH groups, each of which can form an amide bond, two light-absorbing groups are needed to generate one diamine, for example. In this case, a base is generated by cutting one of the amide bonds, each of which binds a base to light-absorbing groups. However, a base still having a light-absorbing group has a large molecular weight, so that the diffusivity of the base becomes poor and thus poor sensitivity could be obtained when used as the base generator. When there is one light-absorbing group per molecule, an excessive amount of relatively-inexpensive base is added to synthesize the base generator; however, when there are two or more light-absorbing groups, it is necessary to add an excessive amount of relatively-expensive material for the light-absorbing groups.

Each of R¹ and R² is preferably an organic group containing no amino group. If an amino group is contained in R¹ and R², the base generator itself becomes a basic substance to promote the reaction of the polymer precursor, and the difference in dissolution contrast between the exposed and unexposed regions could be small.

However, for example, as in the case where an amino group is bound to an aromatic ring that is present in the organic group of R¹ and R², when there is a difference in basicity with a base that is resulted from the exposure to electromagnetic radiation and heating, it is sometimes possible to use the base generator even if an amino group is contained in the organic group of R¹ and R².

As the organic group of R¹ and R², there may be mentioned a saturated or unsaturated alkyl group, a saturated or unsaturated cycloalkyl group, an aryl group, an aralkyl group and a saturated or unsaturated halogenated alkyl group, for example. These organic groups may contain a substituent or a bond other than a hydrocarbon group, such as a heteroatom, and they may be linear or branched.

When R¹ and R² are organic groups, they are generally monovalent organic groups. However, for example, when they form a cyclic structure described below, they may be organic groups which are divalent or more.

Also, R¹ and R² may be bound to form a cyclic structure.

The cyclic structure may be a saturated or unsaturated alicyclic hydrocarbon, a heterocyclic ring, a condensed ring, or a structure comprising a combination of two or more kinds selected from the group consisting of the alicyclic hydrocarbon, heterocyclic ring and condensed ring.

The bond other than a hydrocarbon group in the organic group of R¹ and R² is not particularly limited, and examples of the bond include an ether bond, a thioether bond, a carbonyl bond, a thiocarbonyl bond, an ester bond, an urethane bond, an imino bond (such as —N═C(—R)— or —C(═NR)— wherein R is a hydrogen atom or a monovalent organic group), a carbonate bond, a sulfonyl bond, a sulfinyl bond and an azo bond.

From the viewpoint of heat resistance, preferred are an ether bond, a thioether bond, a carbonyl bond, a thiocarbonyl bond, an ester bond, an urethane bond, an imino bond (such as —N═C(—R)— or —C(═NR)— wherein R is a hydrogen atom or a monovalent organic group), a carbonate bond, a sulfonyl bond and a sulfinyl bond.

The substituent other, than a hydrocarbon group in the organic group of R¹ and R² is not particularly limited as long as the effects of the present invention are not deteriorated. Examples of the substituent include a halogen atom, a hydroxyl group, a mercapto group, a sulfide group, a cyano group, an isocyano group, a cyanato group, an isocyanato group, a thiocyanato group, an isothiocyanato group, a silyl group, a silanol group, an alkoxy group, an alkoxycarbonyl group, a carbamoyl group, a thiocarbamoyl group, a nitro group, a nitroso group, a carboxyl group, a carboxylate group, an acyl group, an acyloxy group, a sulfino group, a sulfo group, a sulfonato group, a phosphino group, a phosphinyl group, a phosphono group, a phosphonato group, a hydroxyimino group, a saturated or unsaturated alkyl ether group, a saturated or unsaturated alkylthioether group, an arylether group, an arylthioether group, an amino group (such as —NH2, —NHR or —NRR′ wherein R and R′ are each independently a hydrocarbon group) and an ammonia group. A hydrogen contained in the above-mentioned substituent may be replaced by a hydrocarbon group. Moreover, a hydrocarbon group contained in the above-mentioned substituent may be linear, branched or cyclic.

Among them, preferred are a halogen atom, a hydroxyl group, a mercapto group, a sulfide group, a cyano group, an isocyano group, a cyanato group, an isocyanato group, a thiocyanato group, an isothiocyanato group, a silyl group, a silanol group, an alkoxy group, an alkoxycarbonyl group, a carbamoyl group, a thiocarbamoyl group, a nitro group, a nitroso group, a carboxyl group, a carboxylate group, an acyl group, an acyloxy group, a sulfino group, a sulfo group, a sulfonato group, a phosphino group, a phosphinyl group, a phosphono group, a phosphonato group, a hydroxyimino group, a saturated or unsaturated alkyl ether group, a saturated or unsaturated alkylthioether group, an arylether group and an arylthioether group.

The thus-generated base is NHR¹R², so that there may be mentioned a primary amine, a secondary amine or a heterocyclic compound. Each of the amines includes an aliphatic amine and an aromatic amine. The heterocyclic compound herein refers to NHR¹R² that has a cyclic structure and has aromaticity. A nonaromatic heterocyclic compound, which is not an aromatic heterocyclic compound, is considered as an alicyclic amine herein and included in aliphatic amines.

As a primary aliphatic amine in the thus-generated NHR¹R², there may be mentioned methylamine, ethylamine, propylamine, isopropylamine, n-butylamine, sec-butylamine, tert-butylamine, pentylamine, isoamylamine, tert-pentylamine, cyclopentylamine, hexylamine, cyclohexylamine, heptylamine, cycloheptanamine, octylamine, 2-octanamine, 2,4,4-trimethylpentane-2-amine and cyclooctylamine, for example.

As a primary aromatic amine in the thus-generated NHR¹R², there may be mentioned aniline, 2-aminophenol, 3-aminophenol and 4-aminophenol, for example.

As a secondary aliphatic amine in the thus-generated NHR¹R², there may be mentioned dimethylamine, diethylamine, dipropylamine, diisopropylamine, dibutylamine, ethylmethylamine, aziridine, azetidine, pyrrolidine, piperidine, azepane, azocane, methylaziridine, dimethylaziridine, methylazetidine, dimethylazetidine, trimethylazetidine, methylpyrrolidine, dimethylpyrrolidine, trimethylpyrrolidine, tetramethylpyrrolidine, methylpiperidine, dimethylpiperidine, trimethylpiperidine, tetramethylpiperidine and pentamethylpiperidine, for example. Among them, preferred is an alicyclic amine.

As a secondary aromatic amine in the thus-generated NHR¹R², there may be mentioned methylaniline, diphenylamine and N-phenyl-1-naphthylamine. As an aromatic heterocyclic compound which has one NH group that can an amide bond, from the viewpoint of basicity, it is preferable that the compound has an imino bond (such as —N═C(—R)— or —C(═NR)— wherein R is a hydrogen atom or a monovalent organic group) in the molecule, and there may be mentioned imidazole, purine, triazole and derivatives thereof.

Thermophysical properties and basicity of the thus-generated base vary depending on the substituent introduced to the position of R¹ and R².

A basic substance with larger basicity provides more effective catalytic action such as reducing the reaction initiation temperature at which the polymer precursor is reacted into a final product. It is thus possible, by the addition of a small amount of the basic substance, to cause the reaction into a final product at a lower temperature. In general, a secondary amine is larger in basicity than a primary amine and provides a large catalytic effect.

An aliphatic amine is more preferable than an aromatic amine since it has larger basicity.

It is preferable that the base generated in the present invention is a secondary amine and/or a heterocyclic compound because, in this case, the base generator becomes more highly sensitive. This is supposed to be because active hydrogen is lost at the amide bond site by using a secondary amine or heterocyclic compound; therefore, there is a change in electron density and thus an increase in isomerization sensitivity.

From the viewpoint of thermophysical properties and basicity of the base to be eliminated, the organic group of R¹ and R² are each independently an organic group preferably having 1 to 20 carbon atoms, more preferably 1 to 12 carbon atoms, and particularly preferably 1 to 8 carbon atoms.

It is especially preferable that the structure of the thus-generated secondary amine and/or heterocyclic compound is represented by the following formula (4):

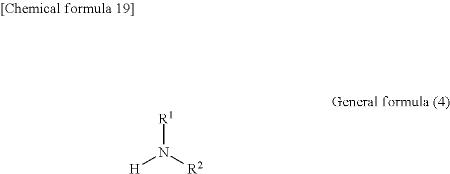

wherein R¹ and R² are each independently an organic group which is an alkyl group that has 1 to 20 carbon atoms and that may have a substituent, or a cycloalkyl group that has 4 to 22 carbon atoms and that may have a substituent; R¹ and R² may be the same or different; and R¹ and R² may be bound to form a cyclic structure which may contain a heteroatom.

In R¹ and R² of the formula (4), the alkyl group may be linear or branched. It is more preferable that the alkyl group is one having 1 to 12 carbon atoms and the cycloalkyl group is one having 4 to 14 carbon atoms. Also preferred is an alicyclic amine in which R¹ and R² are bound to form a cyclic structure that has 4 to 12 carbon atoms and that may have a substituent. Also, a heterocyclic compound in which R¹ and R² are bound to form a cyclic structure that has 2 to 12 carbon atoms and that may have a substituent, is preferred.

In the general formulae (1), (1′), (2), (2′) and (3), R³ and R⁴ are each independently any one of the group consisting of a hydrogen, a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group and an organic group, and R³ and R⁴ may be the same or different.

From the viewpoint of ease of achieving high sensitivity, it is preferable that each of R³ and R⁴ is a hydrogen.

In the present invention, particularly in the case where at least one of R³ and R⁴ is not a hydrogen and is the above-specified functional group, compared with the case where both of R³ and R⁴ are hydrogens, the solubility of the base generator of the present invention in organic solvents or the affinity of the same for polymer precursors is further increased. For example, in the case where at least one of R³ and R⁴ is an organic group such as an alkyl group or aryl group, there is an increase in the solubility in organic solvents. In the case where at least one of R³ and R⁴ is a halogen such as a fluorine, there is an increase in the affinity for polymer precursors containing a halogen such as a fluorine. In the case where at least one of R³ and R⁴ has a silyl group or silanol group, there is an increase in the affinity for polysiloxane precursor. As just described, the solubility of the base generator of the present invention in a desired organic solvent or the affinity of the same for a desired polymer precursor is increased by appropriately introducing a substituent to R³ and/or R⁴ depending on the desired organic solvent or polymer precursor.

As the halogen, there may be mentioned fluorine, chlorine and bromine.

The monovalent organic group is not particularly limited as long as the effects of the present invention are not deteriorated. Examples thereof include a saturated or unsaturated alkyl group, a saturated or unsaturated cycloalkyl group, an aryl group, an aralkyl group, a saturated or unsaturated halogenated alkyl group, a cyano group, an isocyano group, a cyanato group, an isocyanato group, a thiocyanato group, an isothiocyanato group, an alkoxy group, an alkoxycarbonyl group, a carbamoyl group, a thiocarbamoyl group, a carboxyl group, a carboxylate group, an acyl group, an acyloxy group and a hydroxyimino group. These organic groups may contain a substituent or a bond other than a hydrocarbon group, such as a heteroatom, and they may be linear or branched. When R³ and R⁴ are organic groups, they are generally monovalent organic groups.

As the bond other than a hydrocarbon group and the substituent other than a hydrocarbon group in the organic group of R³ and R⁴, there may be used those that are the same as the bond other than a hydrocarbon group and the substituent other than a hydrocarbon group in the organic group of R¹ and R².

Each of R³ and R⁴ may be a hydrogen atom. In the case of having a substituent, at least one of R³ and R⁴ is preferably one selected from the group consisting of: an alkyl group having 1 to 20 carbon atoms, such as a methyl group, ethyl group or propyl group; a cycloalkyl group having 4 to 23 carbon atoms, such as a cyclopentyl group or cyclohexyl group; a cycloalkenyl group having 4 to 23 carbon atoms, such as a cyclopentenyl group or cyclohexenyl group; an aryloxyalkyl group (—ROAr group) having 7 to 26 carbon atoms, such as a phenoxymethyl group, 2-phenoxyethyl group or 4-phenoxybutyl group; an aralkyl group having 7 to 20 carbon atoms, such as a benzyl group or 3-phenylpropyl group; an alkyl group having a cyano group and 2 to 21 carbon atoms, such as a cyanomethyl group or β-cyanoethyl group; an alkyl group having a hydroxyl group and 1 to 20 carbon atoms, such as a hydroxymethyl group; an alkoxy group having 1 to 20 carbon atoms, such as a methoxy group or ethoxy group; an amide group having 2 to 21 carbon atoms, such as an acetamide group or benzenesulfonamide group (C₆H₅SO₂NH₂—); an alkylthio group, (—SR group) having 1 to 20 carbon atoms, such as a methylthio group or ethylthio group; an acyl group having 1 to 20 carbon atoms, such as an acetyl group or benzoyl group; an ester group (—COOR group or —OCOR group) having 2 to 21 carbon atoms, such as a methoxycarbonyl group or acetoxy group; an aryl group having 6 to 20 carbon atoms, such as a phenyl group, naphthyl group, biphenyl group or tolyl group; an aryl group having 6 to 20 carbon atoms with substitution of an electron donating group and/or an electron attracting group; a benzyl group with substitution of an electron donating group and/or an electron attracting group; a cyano group; and a methylthio group (—SCH₃). The alkyl sites may be linear, branched or cyclic.

As the substituent that the base generator of the present invention may have or as R⁵, R^{5′} or R^{5″} of the general formula (2′), (2) or (3), respectively, there may be mentioned a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group, an amino group, an ammonio group or an organic group. Two or more substituents may be the same or different.

Two or more of the substituents that the base generator of the present invention may have or two or more of R⁵s, R^{5′}s or R^{5″}s of the general formula (2), (2′) or (3), respectively, can be bound to form a cyclic structure which may contain a heteroatom. The organic group of R⁵, R^{5′} or R^{5″} of the general formula (2), (2′) or (3), respectively, is generally a monovalent organic group; however, for example, in the case where the below-described cyclic structure is formed, the organic group can be an organic group which is divalent or more.

When the substituent that the base generator of the present invention may have or R⁵, R^{5′} or R^{5″} of the general formula (2), (2′) or (3), respectively, is a halogen or an organic group, there may be used those that are the same as the examples listed above in connection with R³ and R⁴.

Two or more of the substituents that the base generator of the present invention may have or two or more of R⁵s, R^{5′} or R^{5″}s of the general formula (2), (2′) or (3), respectively, may be bound to form a cyclic structure.

The cyclic structure may be a saturated or unsaturated alicyclic hydrocarbon, a heterocyclic ring, a condensed ring or a structure comprising a combination of two or more kinds selected from the group consisting of them. For example, two or more of R⁵s of the general formula (2) may be bound to form a condensed ring such as naphthalene, anthracene, phenanthrene or indene, sharing atoms of the benzene ring to which R⁵s are bound.

In the present invention, because two or more partial structures each represented by the following general formula (1) are contained per molecule, one partial structure represented by the general formula (1) exerts at least an effect of having a substituent on the other partial structure, resulting in an increase in sensitivity, so that the base generator of the present invention does not necessarily needed to have a substituent separately.

However, by introducing a substituent as described above to the base generator of the present invention, it is also possible to adjust the wavelength of a light that the base generator absorbs, or to make the base generator absorb a desired wavelength. For example, by incorporating a substituent that can elongate the conjugated chain of an aromatic ring, it is possible to shift the absorption wavelength to a longer wavelength side. It is also possible to increase the solubility or the compatibility with the polymer precursor to be combined. Thereby, it is possible to increase the sensitivity of the photosensitive resin composition, considering the absorption wavelength of the polymer precursor to be combined.

As a guideline for determining what substituent can be introduced to shift the absorption wavelength to a desired wavelength side or for selecting chemical structure X, “Interpretation of the Ultraviolet Spectra of Natural Products” (A. I. Scott 1964) and tables mentioned in “Spectrometric Identification of Organic Compounds, Fifth Edition” (R. M. Silverstein 1993) can be used.

Preferred as the substituent that the base generator of the present invention may have or as R⁵, R^{5′} or R^{5″} of the general formula (2), (2′) or (3), respectively are an alkyl group having 1 to 20 carbon atoms, a cycloalkyl group having 4 to 23 carbon atoms, a cycloalkenyl group having 4 to 23 carbon atoms, an aryloxyalkyl group having 7 to 26 carbon atoms (—ROAr group), an aralkyl group having 7 to 20 carbon atoms, an alkyl group having a cyano group and 2 to 21 carbon atoms, an alkyl group having a hydroxyl group and 1 to 20 carbon atoms, an alkoxy group having 1 to 20 carbon atoms, an amide group having 2 to 21 carbon atoms, an alkylthio group having 1 to 20 carbon atoms (—SR group), an acyl group having 1 to 20 carbon atoms, an ester group having 2 to 21 carbon atoms, an aryl group having 6 to 20 carbon atoms, an aryl group having 6 to 20 carbon atoms with substitution of an electron-donating group and/or an electron-attracting group, a benzyl group with substitution of an electron-donating group and/or an electron-attracting group, a cyano group and a methylthio group (—SCH₃). The alkyl sites may be linear, branched or cyclic.

It is also preferable that two or more of R⁵s of the general formula (2) are bound to form a condensed ring such as naphthalene, anthracene, phenanthrene or indene, sharing the atoms of the benzene ring to which R⁵s are bound, so that the absorption wavelength of the base generator is shifted to a longer wavelength side.

It is also preferable that the substituent that the base generator of the present invention may have or R⁵, R^{5′} or R^{5″} of the general formula (2), (2′) or (3), respectively, is a hydroxyl group, so that compared to a compound which contains no other hydroxyl group than the partial structure represented by the general formula (1), the solubility of the base generator in basic aqueous solutions or the like can be increased, and the absorption wavelength of the same can be shifted to a longer wavelength side. It is particularly preferable that an ortho position of (—CR⁴═CR³—(C═O)—NR¹R²) of the partial structure represented by the general formula (1) is separately substituted with a phenolic hydroxyl group because there is an increase in the number of reaction sites which are reacted when cyclization of a compound isomerized to a cis isomer takes place, so that the compound is likely to be cyclized.

The partial structure represented by the general formula (1) has a geometric isomer; however, it is preferable to use only a trans isomer as the structure represented by the chemical formula (1). However, there is a possibility that a cis isomer (geometric isomer) is mixed therewith during synthesis and purification processes, storage, etc., and in this case, a mixture of the trans and cis isomers can be used. From the point of view that it is possible to increase the dissolution contrast, the percentage of the cis isomer is preferably less than 10%.

The base generator which comprises a compound having two or more partial structures each represented by the general formula (1) per molecule preferably has a 5% weight loss temperature (a temperature at which there is a weight decrease of 5% from the initial weight by heating) of 100° C. or more, more preferably 200° C. or more. In the case of using a polyimide or polybenzoxazole precursor, it is needed to use a high-boiling solvent such as N-methyl-2-pyrrolidone to form a coating film. However, in the case where the base generator has such a high 5% weight loss temperature, it is possible to form a coating film in a drying condition which can minimize the influence of a residual solvent. Therefore, it is possible to prevent a decrease in the dissolution contrast between the exposed and unexposed regions, which is due to the influence of the residual solvent.

In the present invention, “5% weight loss temperature” is a temperature at which, when measured for weight decrease with a thermogravimetric analyzer, a sample shows a weight decrease of 5% from the initial weight (that is, a temperature at which the weight of the sample is 95% of the initial weight).

It is also preferable that no impurities derived from the base generator of the present invention remain in a product produced by using the photosensitive resin composition of the present invention. Therefore, it is preferable that the base generator of the present invention is decomposed or volatilized in a heating process after development (for example, in the case where the polymer combined is a polyimide precursor, in an imidization process). In particular, the base generator of the present invention preferably has a 5% weight loss temperature of 350° C. or less, more preferably 300° C. or less.

The 5% weight loss temperature of the base generator can be controlled by appropriately selecting substituents R³, R⁴ and R⁵.

The base thus generated preferably has a boiling point of 25° C. or more for ease of handling at room temperature. If the boiling point of the base is not 25° C. or more, an amine thus generated is likely to evaporate from the coating film formed from the photosensitive resin composition especially at the time of drying the film, which could result in difficulty in handling. In the case of using the thus-generated base as a curing accelerator that will not remain in the film, it is preferable that the thus-generated base preferably shows a weight decrease of 80% or more at 350° C., so that it is easy to prevent the base from remaining in the polymer after curing. However, in the case of using the thus-generated base as a crosslinking or curing agent which will remain in the film, the above-described weight decrease of the thus-generated base is not a problem.

In the case of using the base generator of the present invention, the heating temperature for generating a base is appropriately determined depending on the polymer precursor to be combined or on the intended purpose, and it is not particularly limited. The heating can be heating at a temperature of the environment where the base generator is placed (e.g., room temperature) and in this case, bases are gradually generated. Bases can be also generated by heat that is produced as a by-product of the exposure to electromagnetic radiation, so that heating may be substantially performed at the same time by the heat produced as the by-product. To increase the reaction rate and efficiently generate a base, the heating temperature for generating a base is preferably 30° C. or more, more preferably 60° C. or more, still more preferably 100° C. or more, and particularly preferably 120° C. or more. However, the suitable heating temperature is not limited thereto because the unexposed region can be cured by heating at 60° C. or more for example, depending on the type of the polymer precursor used in combination.

To prevent the base generator of the present invention from decomposition other than base generation, the base generator is preferably heated at 300° C. or less.

The base generator which comprises a compound having two or more partial structures each represented by the general formula (1) per molecule generates a base only by exposure to electromagnetic radiation; however, base generation is accelerated by heating the base generator appropriately. Therefore, for efficient base generation, in the case of using the base generator of the present invention, heating is performed after or at the same time as exposure. Exposure and heating may be performed alternately. The most efficient method is heating at the same time as the exposure.

As the method for synthesizing the base generator which comprises a compound having two or more partial structures each represented by the general formula (1) per molecule, there may be mentioned the following methods, for example.

First, an aldehyde derivative to which the intended substituent is introduced, is synthesized. Next, a Wittig, Knoevenagel or Perkin reaction is performed on the aldehyde derivative to synthesize an acid derivative to which the intended substituent is introduced. Among them, Wittig reaction is preferred because it is easy to selectively obtain a trans isomer by the reaction. Then, the target product can be obtained by the condensation reaction between the acid derivative to which the intended substituent is introduced and an appropriately selected amide or basic substance.

For example, the aldehyde to which the intended substituent is introduced can be synthesized by performing a Duff or Vilsmeier-Haack reaction on a phenol having the corresponding substituent. The phenols having the corresponding substituent are commercially available as phenol derivatives which are linked by various linking groups and are materials for functional polymer. Also for example, in the case of being linked by an ether bond, an aldehyde derivative to which the intended substituent is introduced can be synthesized by performing a general ether synthesis method such as Williamson reaction on dihydroxybenzaldehyde.

Also for example, as the method for synthesizing a polymer having a structure represented by the general formula (2-4), for example, there may be mentioned a method for producing a polymer by using an amide having a polymerizable reactive group and a partial structure represented by the general formula (1) as a monomer and polymerizing the monomer according to the reaction system of the polymerizable reaction group, such as radical polymerization, cationic polymerization, anionic polymerization, ring-opening polymerization, polycondensation, polyaddition, addition-condensation or transition metal-catalyzed polymerization. Examples of the polymerizable reactive group include an α,β-ethylenically unsaturated group (such as a vinyl group or (meth)acryloyl group), an alkoxysilane group, an epoxy group and an oxetane group. As the polymerizable reactive group, one or two or more kinds of polymerizable reactive group may be used. For example, in the case of using diester and diol as polymerizable reactive groups, due to polycondensation, the resulting product has a structure in which base generators are bound to side chains of a polyester. In the case of using diisocyanate and ethylene glycol as polymerizable reactive groups, due to polycondensation, the resulting product has a structure in which base generators are bound to side chains of a polyurethane.

It is also possible to produce the amide by such a method that, instead of using the amide having a polymerizable reactive group and a partial structure represented by the general formula (1), a phenol derivative to which a polymerizable reactive group and, as needed, a substituent are introduced, or an aldehyde derivative to which a polymerizable reactive group and, as needed, a substituent are introduced, is used as a monomer to produce a polymer; the phenol derivative is transformed into an aldehyde derivative and the aldehyde derivative is transformed into an acid derivative in the same manner as above; then, the amide is obtained by the condensation reaction of the acid derivative with the basic substance.

Examples of different polymer synthesis methods include the following method: first, a monomer to which a polymerizable reactive group and, as needed, an active group are introduced is polymerized in the same manner as above to synthesis a polymer to which an active group is introduced, or a polymer having an active group (such as a polyvalent carboxylic acid, a polyvalent hydroxyl group, a polyvalent amine, a polyvalent isocyanate or an epoxy resin) is prepared, and the active group of the polymer is reacted with any one selected from the group consisting of the following: an amide having an active group reactive with the reactive group and a partial structure represented by the general formula (1); an aldehyde derivative having an active group reactive with the active group and; a phenol derivative having an active group reactive with the active group. In the case of using the phenol derivative or aldehyde derivative, in the same manner as above, the phenol derivative is transformed into an aldehyde derivative and the aldehyde derivative is transformed into an acid derivative, and then the amide is obtained by the condensation reaction of the acid derivative with the basic substance.

Examples of the combination of usable active groups include a combination of a carboxyl group and a hydroxyl, amino or epoxy group, a combination of an epoxy group and an amino, hydroxyl or carboxyl group, and a combination of an isocyanate group and a hydroxyl group. Any of the combinations can be bound to a polymer as long as one active group is contained in a polymer and the other active group is introduced to a monomer such as an amide or aldehyde derivative as a substituent.

In the present invention, the synthesis method is appropriately selected, depending on the substituent to be introduced or the binding method.

In the case of introducing a substituent to R₄ of the formula (1), first, hydroxyphenyl-(C═O)—R₄ to which the intended substituent is introduced (for example, when R₄ is a methyl group, 2′-hydroxyphenyl methyl ketone to which the intended substituent is introduced) is synthesized. In the case of introducing a substituent only to R₃ of the formula (1), first, a hydroxybenzaldehyde to which the intended substituent is introduced is synthesized, and a Wittig reaction is performed on the hydroxybenzaldehyde using, for example, 1-ethoxycarbonylethylidene-triphenylphosphorane as a reagent for the Wittig reaction to synthesize an acid derivative in which a methyl group is introduced to R₃. The reagent for the Wittig reaction is appropriately selected depending on the substituent which is needed to be introduced to R₃. For example, in the case of an acetyl group, 3-oxo-2-(triphenyl-phosphanylidene)-butyric acid ethyl ester or the like can be used. Then, using the thus-obtained acid derivative, the target product can be obtained in the same manner as above.

The base generator of the present invention, which comprises a compound having two or more partial structures each represented by the following general formula (1) per molecule, is needed to have absorption at at least a part of exposure wavelengths so that the base generator can sufficiently fulfill its base generation function for reacting the polymer precursor into a final product. The wavelengths of a high pressure mercury lamp, which are a general exposing source, are 365 nm, 405 nm and 436 nm. Therefore, the base generator of the present invention preferably has absorption at least one of electromagnetic radiation wavelengths of 365 nm, 405 nm and 436 nm. This is preferable because the types of applicable polymer precursors are further increased in this case.

The base generator of the present invention preferably has a molar absorption coefficient of 100 or more at an electromagnetic radiation wavelength of 365 nm, or a molar absorption coefficient of 1 or more at 405 nm, so that the types of applicable polymer precursors are further increased.

The fact that the base generator of the present invention has absorption in the above-described wavelength range, can be proved by dissolving the base generator of the present invention in a solvent having no absorption in the above wavelength range (e.g., acetonitrile) so as to reach a concentration of 1×10⁻⁴ mol/L or less (it is normally about 1×10⁻⁴ mol/L to 1×10⁻⁵ mol/L and may be appropriately adjusted to reach an appropriate absorption wavelength) and then measuring the absorbance with an ultraviolet-visible spectrophotometer (such as UV-2550 manufactured by Shimadzu Corporation).

The base generator of the present invention preferably has a molecular weight of 250 to 500,000. Especially in the case where the base generator of the present invention is a polymeric base generator, the base generator preferably has a weight average molecular weight of 500 to 500,000, more preferably 1,000 to 100,000.

The molecular weight of the base generator of the present invention is the molecular weight of the compound itself or, in the case of having a molecular weight distribution such as a polymer, the molecular weight of the base generator is a weight average molecular weight.

The base generator of the present invention has higher sensitivity than conventionally used photobase generators and is thus available for a wide range of applications. Various kinds of photosensitive compositions can be produced by not only combining the base generator with a polymer precursor in which reaction into a final product is promoted by a basic substance or by heating in the presence of a basic substance, which will be described below in detail, but also by combining the same with a compound which has a structure or properties that can be changed by a base such as an acid-base indicator. Such photosensitive compositions can be used as a paint, a printing ink, a sealing agent or an adhesive, or as a material for forming display devices, semiconductor devices, electronic components, microelectromechanical systems (MEMS), optical elements or building materials.

For example, the base generator can be applied to a display such as an image forming medium which comprises an image forming layer that contains at least a photobase generator and an acid-base indicator and that covers or penetrates a substrate, and which forms an image in such a manner that when the image forming layer is exposed to light, the photobase generator generates a base that is reactive with the acid-base indicator, thereby forming an image.

In the case where the base generator of the present invention has a structure in which the polymer skeleton has plurality of structures in pendant form, each of which is the structure shown in the brackets of general formula (2), the base generator can be used as a photosensitive resin or base-generating polymer precursor. For example, when the polymer containing a repeating unit represented by the general formula (2-4) is exposed in pattern, the unexposed region is dissolved in a basic solution such as alkaline aqueous solution because it has a phenolic hydroxyl group, while the exposed region is not dissolved in a basic solution such as alkaline aqueous solution because a coumarin derivative is produced in the region by cyclic reaction and thus the phenolic hydroxyl group disappears. Therefore, as a photosensitive resin, the base generator of the present invention can form a pattern.

Therefore, even if the photosensitive resin composition of the present invention does not separately contain the below-described polymer precursor in which reaction into a final product is promoted by a basic substance or by heating in the presence of a basic substance, the photosensitive resin composition comprises a polymer having a repeating unit represented by the following general formula (2-4) as an essential component and can be used as a photosensitive resin composition or as a polymer precursor composition. In the polymer precursor composition or photosensitive resin composition, the base-generating polymer precursor of the present invention may be 100% by weight of the total solid content of the composition. As needed, the photosensitive resin composition or polymer precursor composition may contain other photosensitive component, sensitizer, base amplifier and solvent as described below, and other component.

### <Photosensitive Resin Composition>

The photosensitive resin composition of the present invention comprises a polymer precursor in which reaction into a final product is promoted by a basic substance or by heating in the presence of a basic substance, and the base generator of the present invention which comprises a compound having two or more partial structures each represented by the following general formula (1) per molecule and generates a base by exposure to electromagnetic radiation and heating:

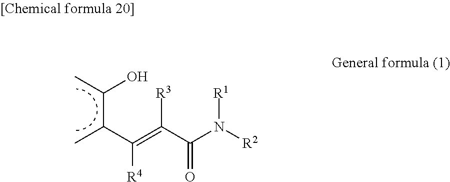

wherein R and R² are each independently a hydrogen or an organic group and may be the same or different; at least one of R¹ and R² is an organic group; R¹ and R² may be bound to form a cyclic structure which may contain a heteroatom but does not contain an amide bond; R³ and R⁴ are each independently a hydrogen, a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group or an organic group and may be the same or different.

As described above, the base generator of the present invention has the above-specified structure. Therefore, by exposing the base generator to electromagnetic radiation, (—CR⁴═CR³—C(═O)—) is isomerized into a cis isomer. By further heating the same, a base (NHR¹R²) is generated. Moreover, when generating a base, the partial structure represented by the formula (1) is cyclized. As a result, a phenolic hydroxyl group is lost to decrease the solubility in a developer that is a basic aqueous solution.

In the polymer precursor, reaction into a final product is promoted by the action of the basic substance generated from the base generator.

Due to such a change in solubility of the polymer precursor and base generator, in the photosensitive resin composition of the present invention, a difference in solubility occurs between the exposed and unexposed regions, that is, the dissolution contrast is increased, so that pattern formation is possible.

As described above, the base generator of the present invention has higher sensitivity than conventional photobase generators, so that the photosensitive resin composition of the present invention is highly sensitive. Also, a wide range of polymer precursors can be applied to the photosensitive resin composition of the present invention, so that the photosensitive resin composition can be widely used in areas where the characteristics of the composition can be utilized, such as the change in solubility of the polymer precursor and base generator. For example, the photosensitive resin composition of the present invention can be suitably used in areas where the characteristics of a photosensitive polyimide precursor resin composition and an imidized product thereof can be utilized. According to the present invention, the dissolution contrast is increased by the change in solubility of the polymer precursor and base generator, so that a polyimide precursor which originally has large solubility in developers can be also used suitably in the present invention.

Hereinafter, the components of the photosensitive resin composition of the present invention will be described. A base generator which can be used for the photosensitive resin composition of the present invention will not be described since, as the base generator, one which is similar to the base generator of the present invention can be used. Accordingly, the polymer precursor and other components that can be contained in the composition as needed, will be described in order.

As the base generator and polymer precursor, only one kind may be used, or a mixture of two or more kinds may be used.

### <Polymer Precursor>

The polymer precursor used for the photosensitive resin composition of the present invention refers to a substance which is finally reacted into a polymer with target properties by a reaction. Examples of the reaction include an intermolecular reaction and an intramolecular reaction. The polymer precursor itself may be a relatively low molecular weight compound or a high molecular weight compound.

The polymer precursor of the present invention is a compound in which reaction into a final product is promoted by a basic substance or by heating in the presence of a basic substance. Examples of the embodiment in which reaction into a final product is promoted in the polymer precursor by a basic substance or by heating in the presence of a basic substance include not only an embodiment in which the polymer precursor is reacted into a final product only by the action of a basis substance, but also an embodiment in which the reaction temperature of the polymer precursor at which the polymer precursor is reacted into a final product by the action of a basic substance is lowered compared to the case without the action of a basic substance.

In the case where there is such a reaction temperature difference due to the presence or absence of a basic substance, by utilizing the reaction temperature difference and heating at an appropriate temperature at which only the polymer precursor coexisting with the basic substance is reacted into a final product, only the polymer precursor coexisting with the basic substance is reacted into a final product, and the solubility of the polymer precursor in a solvent such as a developer is changed. Therefore, the solubility of the polymer precursor in the solvent can be changed by the presence or absence of the basic substance, so that patterning by development using the solvent as a developer is possible.

As the polymer precursor of the present invention, any polymer precursor can be used without particular limitation as long as it can be reacted into a final product by the basis substance as described above or by heating in the presence of such a basic substance. Typical examples of such a polymer precursor will be described below; however, the polymer precursor of the present invention is not limited thereto.

[Polymer Precursor which is Reacted into Polymer by Intermolecular Reaction]

Examples of the polymer precursor which is reacted into a target polymer by an intermolecular reaction include a compound and polymer which have a reactive substituent and cause a polymerization reaction, or a compound and polymer which cause a reaction to form a bond between molecules (crosslinking reaction). Examples of the reactive substituent include an epoxy group, an oxetane group, a thiirane group, an isocyanate group, a hydroxyl group and a silanol group. Examples of the polymer precursor include a compound which causes hydrolysis and polycondensation between molecules, and examples of the reactive substituent include —SiX of polysiloxane precursor, wherein X is a hydrolysable group selected from the group consisting of an alkoxy group, an acetoxy group, an oxime group, an enoxy group, an amino group, an aminooxy group, an amide group and a halogen.

Examples of the compound which has a reactive substituent and causes a polymerization reaction include a compound having one or more epoxy groups, a compound having one or more oxetane groups, and a compound having one or more thiirane groups.

Examples of the polymer which has a reactive substituent and causes a polymerization reaction include a polymer having two or more epoxy groups (epoxy resin), a polymer having two or more oxetane groups, and a polymer having two or more thiirane groups. Among them, the compound and polymer having the epoxy group(s) will be described below in detail. However, the compounds and polymers having the oxetane group(s) and those having the thiirane group(s) can be used similarly to them.

### (Compound and Polymer Having Epoxy Group)

As the compound and polymer having one or more epoxy groups, any conventionally known compound and polymer can be used without particular limitation as long as the compound and polymer have one or more epoxy groups per molecule.

In general, the base generator also functions as a curing catalyst for a compound having one or more epoxy groups per molecule.

In the case of using the compound having one or more epoxy groups per molecule or the polymer having two or more epoxy groups per molecule (epoxy resin), a compound having two or more functional groups per molecule may be used in combination therewith, which are reactive with epoxy groups. Examples of the functional groups which are reactive with epoxy groups include carboxyl groups, phenolic hydroxyl groups, mercapto groups and primary or secondary aromatic amino groups. Considering three dimensional curing properties, the number of the functional groups per molecule of the compound is preferably two or more.

Also, it is preferable to use a polymer which has a weight average molecular weight of 3,000 to 100,000 and in which the functional groups are introduced to a side chain thereof. If the weight average molecular weight is less than 3,000, the strength of a cured film could be decreased; moreover, the surface of the cured film could be tacky and impurities are likely to adhere thereto. It is not preferable that the weight average molecular weight is more than 100,000 because there is a possible increase in viscosity.

An example of the polymer having one or more epoxy groups per molecule is epoxy resin. Examples of the epoxy resin include a bisphenol A type epoxy resin derived from bisphenol A and epichlorohydrin, bisphenol F type epoxy resin derived from bisphenol F and epichlorohydrin, a bisphenol S type epoxy resin, a phenol novolac type epoxy resin, a cresol novolac type epoxy resin, a bisphenol A novolac type epoxy resin, a bisphenol F novolac type epoxy resin, an alicyclic epoxy resin, a diphenyl ether type epoxy resin, a hydroquinone type epoxy resin, a naphthalene type epoxy resin, a biphenyl type epoxy resin, a fluorene type epoxy resin, polyfunctional type epoxy resins such as a trifunctional type epoxy resin an a tetrafunctional type epoxy resin, a glycidyl ester type epoxy resin, a glycidyl amine type epoxy resin, a hydantoin type epoxy resin, an isocyanurate type epoxy resin and a chain aliphatic epoxy resin. These epoxy resins may halogenated or hydrogenated. Commercially available epoxy resin products include, but not limited to, jER coat 828, 1001, 801N, 806, 807, 152, 604, 630, 871, YX8000, YX8034 and YX4000 (manufactured by Japan Epoxy Resins Co., Ltd.), EPICLON 830, EXA835LV, HP4032D and HP820 (manufactured by DIC Corporation), EP4100 series, EP4000 series and EPU series (manufactured by ADEKA Corporation), CELLOXIDE series (2021, 2021E, 2083, 2085, 3000, etc.), EPOLEAD series and EHPE series (manufactured by DAICEL Chemical Industries, Ltd.), YD series, YDF series, YDCN series, YDB series and phenoxy resins (polyhydroxy polyethers each synthesized from a bisphenol and an epichlorohydrin and has an epoxy group at both terminals thereof, such as YE series) (manufactured by Nippon Steel Chemical Co., Ltd.), DENACOL series (manufactured by Nagase ChemteX Corporation), and EPOLIGHT series (manufactured by Kyoeisha Chemical Co., Ltd.), for example. These epoxy resins may be used in combination of two or more kinds. Among them, preferred are bisphenol type epoxy resins because, compared to other various kinds of epoxy compounds, bisphenol type epoxy resin products having different molecular weights are widely available and make it possible to optionally set adhesion, reactivity, etc.

An example of the compound which causes a crosslinking reaction between molecules is a combination of a compound having two or more isocyanate groups per molecule and a compound having two or more hydroxyl groups per molecule. An urethane bond is formed between molecules by the reaction of the isocyanate groups with the hydroxyl groups, so that the combination can be reacted into a polymer.

An example of the polymer which causes a crosslinking reaction between molecules is a combination of a polymer having two or more isocyanate groups per molecule (isocyanate resin) and a polymer having two or more hydroxyl groups per molecule (polyol).

It is also possible to use a combination of a compound and polymer, each of which causes a crosslinking reaction between molecules. Examples of such a combination include a combination of a polymer having two or more isocyanate groups per molecule (isocyanate resin) and a compound having two or more hydroxyl groups per molecule, and a combination of a compound having two or more isocyanate groups per molecule and a polymer having two or more hydroxyl groups per molecule (polyol).

### (Compound and Polymer Having Isocyanate Groups)

As the compound and polymer having isocyanate groups, a conventionally known compound and polymer can be used without particularly limited as long as they have two or more isocyanate groups per molecule. Examples of such a compound include low-molecular-weight compounds such as p-phenylene diisocyanate, 2,4-toluene diisocyanate, 2,6-toluene diisocyanate, 1,5-naphthalene diisocyanate and hexamethylene diisocyanate, an oligomer and a polymer which has a weight average molecular weight of 3,000 or more and in which isocyanate groups are present at a side chain or terminal thereof.

### (Compound and Polymer Having Hydroxyl Groups)

In general, the compound and polymer having isocyanate groups are each used in combination with a compound having hydroxyl groups per molecule. As such a compound having hydroxyl groups, any conventionally known compound can be used without particular limitation as long as it has two or more hydroxyl groups per molecule. Examples of such a compound include low-molecular-weight compounds such as ethylene glycol, propylene glycol, glycerin, diglycerin and pentaerythritol, and a polymer which has a weight average molecular weight of 3,000 or more and in which hydroxyl groups are present at a side chain or terminal thereof.

### (Polysiloxane Precursor)

An example of the compound which causes hydrolysis and polycondensation between molecules is a polysiloxane precursor.

Examples of the polysiloxane precursor include an organic silicon compound represented by YₙSiX₍₄₋ₙ₎ (wherein Y is a hydrogen or an alkyl group, fluoroalkyl group, vinyl group or phenyl group which may have a substituent; X is a hydrolysable group selected from the group consisting of an alkoxy group, an acetoxy group, an oxime group, an enoxy group, an amino group, an aminooxy group, an amide group and a halogen; and n is an integer of 0 to 3) and a hydrolyzed polycondensate of the organic silicon compound. Among them, preferred is one represented by the above formula wherein n is an integer of 0 to 2. As the hydrolysable group, preferred is an alkoxy group in terms of the ease of preparing a silica-dispersed oligomer solution and its availability.

The organic silicon compound is not particularly limited and conventionally known organic silicon compounds can be used as the compound. Examples thereof include trimethoxysilane, triethoxysilane, methyltrichlorosilane, methyltrimethoxysilane, methyltriethoxysilane, methyltriisopropoxysilane, methyltri-t-butoxysilane, ethyltribromosilane, ethyltrimethoxysilane, ethyltriethoxysilane, n-propyltriethoxysilane, n-hexyltrimethoxysilane, phenyltrimethoxysilane, phenyltriethoxysilane, tetramethoxysilane, tetraethoxysilane, tetrabutoxysilane, dimethoxydiethoxysilane, dimethyldichlorosilane, dimethyldimethoxysilane, diphenyldimethoxysilane, vinyltrimethoxysilane, trifluoropropyltrimethoxysilane, γ-glycidoxypropylmethyldiethoxysilane, γ-glycidoxypropyltrimethoxysilane, γ-methacryloxypropylmethyldimethoxysilane, γ-aminopropylmethyldimethoxysilane, γ-mercaptopropylmethyldiethoxysilane, γ-mercaptopropyltrimethoxysilane, β-(3,4-epoxycyclohexyl)ethyltrimethoxysilane, fluoroalkylsilane which is known as a fluorine-containing silane coupling agent, hydrolysis-condensation products or hydrolysis-cocondensation products thereof, and mixtures thereof.

[Polymer Precursor which is Reacted into Polymer by Intramolecular Ring Closure Reaction]

Examples of the polymer precursor which is finally reacted into a polymer with target properties by an intermolecular ring closure reaction include a polyimide precursor and a polybenzoxazole precursor. Each of these precursors may be a mixture of two or more polymer precursors synthesized separately.

Hereinafter, the polyimide precursor and polybenzoxazole precursor which are polymer precursors preferred in the present invention will be described. However, the present invention is not limited thereto.

### (Polyimide Precursor)

As the polyimide precursor, a polyamic acid having a repeating unit represented by the following chemical formula (5) is suitably used:

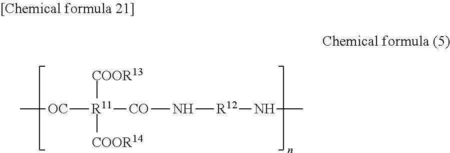

wherein R¹¹ is a tetravalent organic group; R¹² is a divalent organic group; each of R¹³ and R¹⁴ is a hydrogen atom or a monovalent organic group; and n is a natural number of 1 or more.

When R¹³ and R¹⁴ are monovalent organic groups, examples thereof include an alkyl group, an alkenyl group, an alkynyl group, an aryl group and structures comprising these groups and an ether bond, as represented by the formula CₙH₂ₙOCₘH₂ₘ₊₁.

As the polyimide precursor, a polyamic acid such that R¹³ and R¹⁴ are hydrogen atoms, is suitably used from the viewpoint of alkali developing properties.

The tetravalence of R¹¹ only refers to a valence for bonding to acids; however, R¹¹ may have other substituent(s) further. Similarly, the divalence of R¹² refers only to a valence for bonding to amines; however, R¹² may have other substituent(s) further.

Polyamic acid is preferred because it can be obtained only by mixing an acid dianhydride with a diamine in a solution, so that it can be synthesized by a one-step reaction, is easy to synthesize and can be obtained at low cost.

There is such a secondary effect that when the polymer precursor used is a polyamic acid, a low temperature is good enough for imidization to take place due to the catalytic effect of the basic substance, so that it is possible to decrease the final curing temperature to less than 300° C., preferably 250° C. or less.

Conventional polyamic acids have limited applications since the final curing temperature is needed to be 300° C. or more for imidization to take place; however, it is now possible by the present invention to decrease the final curing temperature and thus to use the polyamic acid in a wide range of applications.

A polyamic acid can be obtained by the reaction of an acid dianhydride and a diamine. However, to provide excellent heat resistance and dimensional stability to the finally-obtained polyimide, it is preferable that R¹¹ or R¹² of the chemical formula (5) is an aromatic compound, and it is more preferable that R¹¹ and R¹² of the chemical formula (5) are aromatic compounds. In this case, at R¹¹ of the chemical formula (5), four groups ((—CO—)₂(—COOH)₂) bound to R¹¹ may be bound to the same aromatic ring or different aromatic rings. Similarly, at R¹² of the chemical formula (5), two groups ((—NH—)₂) bound to R¹² may be bound to the same aromatic ring or different aromatic rings.

The polyamic acid represented by the chemical formula (5) may be one comprising a single repeating unit or one comprising two or more kinds of repeating units.

Conventionally known methods can be used as the method for producing the polyimide precursor of the present invention. Examples thereof include, but not limited to, (1) a method for synthesizing a polyamic acid (precursor) from an acid dianhydride and a diamine, and (2) a method for synthesizing a polyimide precursor by the reaction of a carboxylic acid of an ester acid or amide acid monomer with a diamino compound or derivative thereof, the ester acid or amino acid monomer being synthesized by the reaction of an acid dianhydride with a monovalent alcohol, an amino compound, an epoxy compound, or the like.

Examples of the acid dianhydride which are applicable to the reaction for obtaining the polyimide precursor of the present invention include aliphatic tetracarboxylic dianhydrides such as an ethylenetetracarboxylic dianhydride, butanetetracarboxylic dianhydride, cyclobutanetetracarboxylic dianhydride, methylcyclobutanetetracarboxylic dianhydride and cyclopentanetetracarboxylic dianhydride; and aromatic tetracarboxylic dianhydrides such as pyromellitic dianhydride, 3,3′,4,4′-benzophenonetetracarboxylic dianhydride, 2,2′,3,3′-benzophenonetetracarboxylic dianhydride, 2,3′,3,4′-benzophenonetetracarboxylic dianhydride, 3,3′,4,4′-biphenyltetracarboxylic dianhydride, 2,2′,3,3′-biphenyltetracarboxylic dianhydride, 2,3′,3,4′-biphenyltetracarboxylic dianhydride, 2,2′,6,6′-biphenyltetracarboxylic dianhydride, 2,2-bis(3,4-dicarboxyphenyl)propane dianhydride, 2,2-bis(2,3-dicarboxyphenyl)propane dianhydride, bis(3,4-dicarboxyphenyl)ether dianhydride, bis(3,4-dicarboxyphenyl)sulfone dianhydride, 1,1-bis(2,3-dicarboxyphenyl)ethane dianhydride, bis(2,3-dicarboxyphenyl)methane dianhydride, bis(3,4-dicarboxyphenyl)methane dianhydride, 2,2-bis(3,4-dicarboxyphenyl)-1,1,1,3,3,3-hexafluoropropane dianhydride, 2,2-bis(2,3-dicarboxyphenyl)-1,1,1,3,3,3-hexafluoropropane dianhydride, 1,3-bis[(3,4-dicarboxy)benzoyl]benzene dianhydride, 1,4-bis[(3,4-dicarboxy)benzoyl]benzene dianhydride, 2,2-bis{4-[4-(1,2-dicarboxy)phenoxy]phenyl}propane dianhydride,

2,2-bis{4-[3-(1,2-dicarboxy)phenoxy]phenyl}propane dianhydride, bis{4-[4-(1,2-dicarboxy)phenoxy]phenyl}ketone dianhydride, bis{4-[3-(1,2-dicarboxy)phenoxy]phenyl}ketone dianhydride, 4,4′-bis[4-(1,2-dicarboxy)phenoxy]biphenyl dianhydride, 4,4′-bis[3-(1,2-dicarboxy)phenoxy]biphenyl dianhydride, bis{4-[4-(1,2-dicarboxy)phenoxy]phenyl}ketone dianhydride, bis{4-[3-(1,2-dicarboxy)phenoxy]phenyl}ketone dianhydride, bis{4-[4-(1,2-dicarboxy)phenoxy]phenyl}sulfone dianhydride, bis{4-[3-(1,2-dicarboxy)phenoxy]phenyl}sulfone dianhydride, bis{4-[4-(1,2-dicarboxy)phenoxy]phenyl}sulfide dianhydride, bis{4-[3-(1,2-dicarboxy)phenoxy]phenyl}sulfide dianhydride, 2,2-bis{4-[4-(1,2-dicarboxy)phenoxy]phenyl}-1,1,1,3,3,3-hexafluoropropane dianhydride, 2,2-bis{4-[3-(1,2-dicarboxy)phenoxy]phenyl}-1,1,1,3,3,3-hexafluoropropane dianhydride, 2,3,6,7-naphthalenetetracarboxylic dianhydride, 1,1,1,3,3,3-hexafluoro-2,2-bis(2,3- or 3,4-dicarboxyphenyl)propane dianhydride, 1,4,5,8-naphthalenetetracarboxylic dianhydride, 1,2,5,6-naphthalenetetracarboxylic dianhydride, 1,2,3,4-benzenetetracarboxylic dianhydride, 3,4,9,10-perylenetetracarboxylic dianhydride, 2,3,6,7-anthracenetetracarboxylic dianhydride, 1,2,7,8-phenanthrenetetracarboxylic dianhydride, pyridinetetracarboxylic dianhydride, sulfonyldiphthalic anhydride, m-terphenyl-3,3′,4,4′-tetracarboxylic dianhydride, and p-terphenyl-3,3′,4,4′-tetracarboxylic dianhydride. They are used solely or in combination of two or more kinds. Preferred tetracarboxylic dianhydrides are pyromellitic dianhydride, 3,3′,4,4′-benzophenonetetracarboxylic dianhydride, 3,3′,4,4′-biphenyltetracarboxylic dianhydride, 2,2′,6,6′-biphenyltetracarboxylic dianhydride, bis(3,4-dicarboxyphenyl)ether dianhydride and 2,2-bis(3,4-dicarboxyphenyl)-1,1,1,3,3,3-hexafluoropropane dianhydride.

In the case of using, as the acid anhydride used in combination, an acid dianhydride having a fluorine introduced thereto or an acid dianhydride having an alicyclic skeleton, it is possible to control physical properties (e.g., solubility and thermal expansion coefficient) without a large deterioration in transparency. In the case of using a rigid acid dianhydride such as pyromellitic dianhydride, 3,3′,4,4′-biphenyltetracarboxylic dianhydride or 1,4,5,8-naphthalenetetracarboxylic dianhydride, the finally-obtained polyimide is provided with a small linear thermal expansion coefficient; however, there is a tendency that the use inhibits an increase in transparency, so that such a rigid acid dianhydride may be used in combination, paying attention to copolymerization ratio.

Meanwhile, as the amine component, one kind of diamine can be used solely or two or more kinds of diamines can be used in combination. The used diamine component(s) is not limited and examples thereof include aromatic diamines such as p-phenylenediamine, m-phenylenediamine, o-phenylenediamine, 3,3′-diaminodiphenyl ether, 3,4′-diaminodiphenyl ether, 4,4′-diaminodiphenyl ether, 3,3′-diaminodiphenyl sulfide, 3,4′-diaminodiphenyl sulfide, 4,4′-diaminodiphenyl sulfide, 3,3′-diaminodiphenyl sulfone, 3,4′-diaminodiphenyl sulfone, 4,4′-diaminodiphenyl sulfone, 3,3′-diaminobenzophenone, 4,4′-diaminobenzophenone, 3,4′-diaminobenzophenone, 3,3′-diaminodiphenylmethane, 4,4′-diaminodiphenylmethane, 3,4′-diaminodiphenylmethane, 2,2-di(3-aminophenyl)propane, 2,2-di(4-aminophenyl)propane, 2-(3-aminophenyl)-2-(4-aminophenyl)propane, 2,2-di(3-aminophenyl)-1,1,1,3,3,3-hexafluoropropane, 2,2-di(4-aminophenyl)-1,1,1,3,3,3-hexafluoropropane, 2-(3-aminophenyl)-2-(4-aminophenyl)-1,1,1,3,3,3-hexafluoropropane, 1,1-di(3-aminophenyl)-1-phenylethane, 1,1-di(4-aminophenyl)-1-phenylethane, 1-(3-aminophenyl)-1-(4-aminophenyl)-1-phenylethane, 1,3-bis(3-aminophenoxy)benzene, 1,3-bis(4-aminophenoxy)benzene, 1,4-bis(3-aminophenoxy)benzene, 1,4-bis(4-aminophenoxy)benzene, 1,3-bis(3-aminobenzoyl)benzene, 1,3-bis(4-aminobenzoyl)benzene, 1,4-bis(3-aminobenzoyl)benzene, 1,4-bis(4-aminobenzoyl)benzene, 1,3-bis(3-amino-α,α-dimethylbenzyl)benzene, 1,3-bis(4-amino-α,α-dimethylbenzyl)benzene, 1,4-bis(3-amino-α,α-dimethylbenzyl)benzene, 1,4-bis(4-amino-α,α-dimethylbenzyl)benzene, 1,3-bis(3-amino-α,α-ditrifluoromethylbenzyl)benzene, 1,3-bis(4-amino-α,α-ditrifluoromethylbenzyl)benzene, 1,4-bis(3-amino-α,α-ditrifluoromethylbenzyl)benzene, 1,4-bis(4-amino-α,α-ditrifluoromethylbenzyl)benzene, 2,6-bis(3-aminophenoxy)benzonitrile, 2,6-bis(3-aminophenoxy)pyridine, 4,4′-bis(3-aminophenoxy)biphenyl, 4,4′-bis(4-aminophenoxy)biphenyl, bis[4-(3-aminophenoxy)phenyl]ketone, bis[4-(4-aminophenoxy)phenyl]ketone, bis[4-(3-aminophenoxy)phenyl]sulfide, bis[4-(4-aminophenoxy)phenyl]sulfide, bis[4-(3-aminophenoxy)phenyl]sulfone, bis[4-(4-aminophenoxy)phenyl]sulfone, bis[4-(3-aminophenoxy)phenyl]ether, bis[4-(4-aminophenoxy)phenyl]ether, 2,2-bis[4-(3-aminophenoxy)phenyl]propane, 2,2-bis[4-(4-aminophenoxy)phenyl]propane, 2,2-bis[3-(3-aminophenoxy)phenyl]-1,1,1,3,3,3-hexafluoropropane, 2,2-bis[4-(4-aminophenoxy)phenyl]-1,1,1,3,3,3-hexafluoropropane, 1,3-bis[4-(3-aminophenoxy)benzoyl]benzene, 1,3-bis[4-(4-aminophenoxy)benzoyl]benzene, 1,4-bis[4-(3-aminophenoxy)benzoyl]benzene, 1,4-bis[4-(4-aminophenoxy)benzoyl]benzene, 1,3-bis[4-(3-aminophenoxy)-α,α-dimethylbenzyl]benzene, 1,3-bis[4-(4-aminophenoxy)-α,α-dimethylbenzyl]benzene, 1,4-bis[4-(3-aminophenoxy)-α,α-dimethylbenzyl]benzene, 1,4-bis[4-(4-aminophenoxy)-α,α-dimethylbenzyl]benzene, 4,4′-bis[4-(4-aminophenoxy)benzoyl]diphenyl ether, 4,4′-bis[4-(4-amino-α,α-dimethylbenzyl)phenoxy]benzophenone, 4,4′-bis[4-(4-amino-α,α-dimethylbenzyl)phenoxy]diphenyl sulfone, 4,4′-bis[4-(4-aminophenoxy)phenoxy]diphenyl sulfone, 3,3′-diamino-4,4′-diphenoxybenzophenone, 3,3′-diamino-4,4′-dibiphenoxybenzophenone, 3,3′-diamino-4-phenoxybenzophenone, 3,3′-diamino-4-biphenoxybenzophenone, 6,6′-bis(3-aminophenoxy)-3,3,3′,3′-tetramethyl-1,1′-spirobiindan, and 6,6′-bis(4-aminophenoxy)-3,3,3′,3′-tetramethyl-1,1′-spirobiindan; aliphatic amines such as 1,3-bis(3-aminopropyl)tetramethyldisiloxane, 1,3-bis(4-aminobutyl)tetramethyldisiloxane, α,ω-bis(3-aminopropyl)polydimethylsiloxane, α,ω-bis(3-aminobutyl)polydimethylsiloxane, bis(aminomethyl)ether, bis(2-aminoethyl)ether, bis(3-aminopropyl)ether, bis(2-aminomethoxy)ethyl]ether, bis[2-(2-aminoethoxy)ethyl]ether, bis[2-(3-aminoprotoxy)ethyl]ether, 1,2-bis(aminomethoxy)ethane, 1,2-bis(2-aminoethoxy)ethane, 1,2-bis[2-(aminomethoxy)ethoxy]ethane, 1,2-bis[2-(2-aminoethoxy)ethoxy]ethane, ethylene glycol bis(3-aminopropyl)ether, diethylene glycol bis(3-aminopropyl)ether, triethylene glycol bis(3-aminopropyl)ether, ethylenediamine, 1,3-diaminopropane, 1,4-diaminobutane, 1,5-diaminopentane, 1,6-diaminohexane, 1,7-diaminoheptane, 1,8-diaminooctane, 1,9-diaminononane, 1,10-diaminodecane, 1,11-diaminoundecane, and 1,12-diaminododecane; and

alicyclic diamines such as 1,2-diaminocyclohexane, 1,3-diaminocyclohexane, 1,4-diaminocyclohexane, 1,2-di(2-aminoethyl)cyclohexane, 1,3-di(2-aminoethyl)cyclohexane, 1,4-di(2-aminoethyl)cyclohexane, bis(4-aminocyclohexyl)methane, 2,6-bis(aminomethyl)bicyclo[2.2.1]heptane, and 2,5-bis(aminomethyl)bicyclo[2.2.1]heptane. Guanamines include acetoguanamine and benzoguanamine. Also, it is possible to use a diamine which is obtained by substituting part or all of hydrogen atoms of the aromatic ring of any of the above diamines with a substituent selected from the group consisting of a fluoro group, a methyl group, a methoxy group, a trifluoromethyl group and a trifluoromethoxy group.

Furthermore, depending on the intended purpose, any one or two or more of an ethynyl group, a benzocyclobutene-4′-yl group, a vinyl group, an allyl group, a cyano group, an isocyanate group and an isopropenyl group can be introduced to part or all of the hydrogen atoms of the aromatic ring of any of the above diamines as a substituent, the groups serving as a crosslinking point.

The diamine can be selected depending on target properties, and in the case of using a rigid diamine such as p-phenylenediamine, the finally-obtained polyimide is provided with a low expansion coefficient. Examples of the rigid diamine include a diamine in which two amino groups are bound to one aromatic ring, such as p-phenylenediamine, m-phenylenediamine, 1,4-diaminonaphthalene, 1,5-diaminonaphthalene, 2,6-diaminonaphthalene, 2,7-diaminonaphthalene and 1,4-diaminoanthracene.

Moreover, there may be mentioned a diamine in which two or more aromatic rings are connected via a direct bond and two or more amino groups are each bound to the different aromatic rings directly or as a part of a substituent, such as a diamine represented by the following formula (6). Specific examples thereof include benzidine.

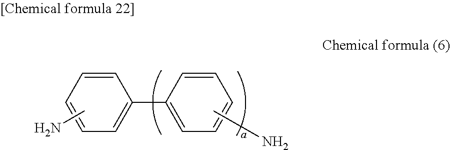

In the formula (6), a is a natural number of 1 or more, and each of the amino groups is bound to the meta- or para-position of the bond between the benzene rings.

Also, there may be used a diamine which is represented by the formula (6) and in which a substituent is present in a position of each benzene ring, the position being not involved in bonding to the other benzene ring and not replaced by an amino group. The substituents are organic groups; however, they may be bound to each other.

Specific examples thereof include 2,2′-dimethyl-4,4′-diaminobiphenyl, 2,2′-ditrifluoromethyl-4,4′-diaminobiphenyl, 3,3′-dichloro-4,4′-diaminobiphenyl, 3,3′-dimethoxy-4,4′-diaminobiphenyl and 3,3′-dimethyl-4,4′-diaminobiphenyl.

In the case of using the finally-obtained polyimide as an optical waveguide or optical circuit component, it is possible to increase the transmittance of the polyimide for electromagnetic radiation at a wavelength of 1 μm or less by introducing a fluorine as a substituent of each aromatic ring.

In the case of using a diamine having a siloxane skeleton (e.g., 1,3-bis(3-aminopropyl)tetramethyldisiloxane) as the diamine, there is a decrease in the elastic modulus of the finally-obtained polyimide and thus a decrease in the glass transition temperature of the same.

From the viewpoint of heat resistance, the diamine selected herein is preferably an aromatic diamine. However, depending on target properties, a diamine other than aromatic diamine (e.g., aliphatic diamine and siloxane diamine) may be used in an amount that is less than 60% by mole, preferably 40% by mole of the whole diamine.

The polyimide precursor can be synthesized as follows, for example: a solution is prepared by dissolving 4,4′-diaminodiphenyl ether (amine component) in an organic polar solvent such as N-methylpyrrolidone; while cooling the solution, an equimolar 3,3′,4,4′-biphenyltetracarboxylic dianhydride is gradually added thereto and stirred, thereby obtaining a polyimide precursor solution.

To provide heat resistance and dimensional stability to the finally-obtained polyimide, the copolymerization ratio of the aromatic acid component and/or the aromatic amine component in the polyimide precursor synthesized as above is preferably as large as possible. In particular, the aromatic acid component is preferably 50% by mole or more, more preferably 70% by mole or more of the acid component constituting the repeating unit of the imide structure; the aromatic amine component is preferably 40% by mole or more, more preferably 60% by mole or more of the amine component constituting the repeating unit of the imide structure; and a wholly aromatic polyimide is particularly preferable.

### <Polybenzoxazole Precursor>

As the polybenzoxazole precursor used in the present invention, a polyamide alcohol having a repeating unit represented by the following chemical formula (5) is suitably used.

The polyamide alcohol can be synthesized by conventionally known methods. For example, it can be obtained by the addition reaction of a dicarboxylic acid derivative (e.g., dicarboxylic acid halide) with a dihydroxydiamine in an organic solvent.

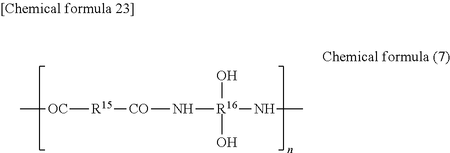

In the chemical formula (7), R¹⁵ is a divalent organic group; R¹⁶ is a tetravalent organic group; and n is a natural number of 1 or more.

The divalence of R¹⁵ refers only to a valence for bonding to acids; however, R may have other substituent(s) further. Similarly, the tetravalence of R¹⁶ refers only to a valence for bonding to amines and hydroxyl groups; however, R¹⁶ may have other substituent(s) further.

To provide excellent heat resistance and dimensional stability to the finally-obtained polybenzoxazole, the polyamide alcohol having a repeating unit represented by the chemical formula (7) is preferably such that R¹⁵ or R¹⁶ of the chemical formula (7) is an aromatic compound, and it is more preferable that R¹⁵ and R¹⁶ of the chemical formula (7) are aromatic compounds. In this case, at R¹⁵ of the chemical formula (7), two groups ((−CO—)₂) bound to R¹⁵ may be bound to the same aromatic ring or different aromatic rings. Similarly, at R¹⁶ of the chemical formula (7), four groups ((—NH—)₂(—OH)₂) bound to R¹⁶ may be bound to the same aromatic ring or different aromatic rings.

The polyamide alcohol represented by the chemical formula (7) may be one comprising a single repeating unit or one comprising two or more kinds of repeating units.

Examples of the dicarboxylic acid or derivative thereof which can be applied to the reaction for obtaining the polybenzoxazole precursor include, but not limited to, phthalic acid, isophthalic acid, terephthalic acid, 4,4′-benzophenone dicarboxylic acid, 3,4′-benzophenone dicarboxylic acid, 3,3′-benzophenone dicarboxylic acid, 4,4′-biphenyldicarboxylic acid, 3,4′-biphenyldicarboxylic acid, 3,3′-biphenyldicarboxylic acid, 4,4′-diphenyl ether dicarboxylic acid, 3,4′-diphenyl ether dicarboxylic acid, 3,3′-diphenyl ether dicarboxylic acid, 4,4′-diphenyl sulfone dicarboxylic acid, 3,4′-diphenyl sulfone dicarboxylic acid, 3,3′-diphenyl sulfone dicarboxylic acid, 4,4′-hexafluoroisopropylidene dibenzoic acid, 4,4′-dicarboxydiphenylamide, 1,4-phenylenediethanoic acid, 1,1-bis(4-carboxyphenyl)-1-phenyl-2,2,2-trifluoroethane, bis(4-carboxyphenyl)tetraphenyldisiloxane, bis(4-carboxyphenyl)tetramethyldisiloxane, bis(4-carboxyphenyl)sulfone, bis(4-carboxyphenyl)methane, 5-t-butylisophthalic acid, 5-bromoisophthalic acid, 5-fluoroisophthalic acid, 5-chloroisophthalic acid, 2,2-bis-(p-carboxyphenyl)propane, 4,4′-(p-phenylenedioxy)dibenzoic acid, 2,6-naphthalenedicarboxylic acid, acid halides thereof, and active esters thereof with hydroxybenzotriazole or the like. They are used solely or in combination of two or more kinds.

Specific examples of the dihydroxydiamine include, but not limited to, 3,3′-dihydroxybenzidine, 3,3′-diamino-4,4′-dihydroxybiphenyl, 4,4′-diamino-3,3′-dihydroxybiphenyl, 3,3′-diamino-4,4′-dihydroxydiphenyl sulfone, 4,4′-diamino-3,3′-dihydroxydiphenyl sulfone, bis-(3-amino-4-hydroxyphenyl)methane, 2,2-bis-(3-amino-4-hydroxyphenyl)propane, 2,2-bis-(3-amino-4-hydroxyphenyl)hexafluoropropane, 2,2-bis-(4-amino-3-hydroxyphenyl)hexafluoropropane, bis-(4-amino-3-hydroxyphenyl)methane, 2,2-bis-(4-amino-3-hydroxyphenyl)propane, 4,4′-diamino-3,3′-dihydroxybenzophenone, 3,3′-diamino-4,4′-dihydroxybenzophenone, 4,4′-diamino-3,3′-dihydroxydiphenyl ether, 3,3′-diamino-4,4′-dihydroxydiphenyl ether, 1,4-diamino-2,5-dihydroxybenzene, 1,3-diamino-2,4-dihydroxybenzene, and 3-diamino-4,6-dihydroxybenzene. They may be used solely or in combination of two or more kinds.

The polymer precursor such as polyimide precursor or polybenzoxazole precursor preferably shows a transmittance of at least 5% or more, more preferably 15% or more for the exposure wavelength when it is formed into a film having a thickness of 1 μm, so that the photosensitive resin composition thus obtained is provided with high sensitivity and a pattern shape that can accurately reproduce a mask pattern is obtained.

The higher the transmittance of the polymer precursor (such as polyimide precursor or polybenzoxazole precursor) for the exposure wavelength, the smaller the loss of electromagnetic radiation. Therefore, a highly sensitive photosensitive resin composition can be obtained.

In the case of using a high pressure mercury lamp, which is a general exposing source, for exposure, the polymer precursor preferably has a transmittance of 5% or more, more preferably 15%, still more preferably 50% or more, for at least one of electromagnetic radiation wavelengths of 436 nm, 405 nm and 365 nm, when it is formed into a film having a thickness of 1 μm.

The polymer precursor such as polyimide precursor or polybenzoxazole precursor has a weight average molecular weight in the range of, although it depends on the intended use, preferably 3,000 to 1,000,000, more preferably 5,000 to 500,000, still more preferably 10,000 to 500,000. When the weight average molecular weight is less than 3,000, a coating or film made of the polymer precursor is not likely to have sufficient strength. Also, low strength is provided to a film formed from a polymer (e.g., polyimide) converted from the polymer precursor by heating treatment or the like. On the other hand, when the weight average molecular weight exceeds 1,000,000, the viscosity of the polymer precursor is increased and the solubility of the same is likely to be decreased; therefore, it is difficult to obtain a coating or film having a smooth surface and uniform thickness.

In the present invention, the molecular weight is a polystyrene-equivalent value obtained by gel permeation chromatography (GPC). It may be the molecular weight of the polymer precursor itself (e.g., polyimide precursor) or may be the molecular weight after a chemical imidization treatment is performed thereon with acetic anhydride or the like.

The solvent used for the synthesis of the polyimide precursor or polybenzoxazole precursor is preferably a polar solvent. Typical examples thereof include N-methyl-2-pyrrolidone, N-acetyl-2-pyrrolidone, N,N-dimethylformamide, N,N-dimethylacetamide, N,N-diethylformamide, N,N-diethylacetamide, N,N-dimethylmethoxyacetamide, dimethylsulfoxide, hexamethylphosphoramide, pyridine, dimethyl sulfone, tetramethylene sulfone, dimethyltetramethylene sulfone, diethylene glycol dimethyl ether, cyclopentanone, γ-butyrolactone and α-acetyl-γ-butyrolactone. They are used solely or in combination of two or more kinds. Besides, a non-polar solvent can be used in combination with the solvent, and examples thereof include benzene, benzonitrile, 1,4-dioxane, tetrahydrofuran, butyrolactone, xylene, toluene and cyclohexanone. These solvents are used as a dispersion medium for raw materials, a reaction control agent, an agent for controlling solvent volatilization from a product, a coating film smoothing agent, etc.

The solubility of the polyamic acid or polybenzoxazole precursor is decreased as the reaction of the same into a final product is promoted by the action of a basic substance. Therefore, when combined with a decrease in solubility which is due to the base generated from the base generator of the present invention, there is an advantage that the dissolution contrast between the exposed and unexposed regions of the photosensitive resin composition of the present invention can be increased further.

### <Other Components>

The photosensitive resin composition of the present invention may be a simple mixture of the base generator of the present invention, one or more kinds of polymer precursors and a solvent. Also, it may be prepared by adding a photo- or heat-curable component, a non-polymerizable binder resin other than the polymer precursor, and other component to the mixture.

Various kinds of all-purpose solvents can be used as the solvent for dissolving, dispersing or diluting the photosensitive resin composition. In the case of using a polyamide acid as the precursor, a solution obtained by the synthesis reaction of the polyamide acid may be used as it is, and the solution may be mixed with other component as needed.

Usable all-purpose solvents include, for example, ethers such as diethyl ether, tetrahydrofuran, dioxane, diethylene glycol dimethyl ether, ethylene glycol diethyl ether, propylene glycol dimethyl ether, propylene glycol diethyl ether and diethylene glycol dimethyl ether; glycol monoethers (so-called cellosolves) such as ethylene glycol monomethyl ether, ethylene glycol monoethyl ether, propylene glycol monomethyl ether, propylene glycol monoethyl ether, diethylene glycol monomethyl ether and diethylene glycol monoethyl ether; ketones such as methyl ethyl ketone, acetone, methyl isobutyl ketone, cyclopentanone and cyclohexanone; esters such as ethyl acetate, butyl acetate, n-propyl acetate, i-propyl acetate, n-butyl acetate, i-butyl acetate, ester acetates of the glycol monoethers (e.g., methyl cellosolve acetate, ethyl cellosolve acetate), propylene glycol monomethyl ether acetate, propylene glycol monoethyl ether acetate, dimethyl oxalate, methyl lactate and ethyl lactate; alcohols such as ethanol, propanol, butanol, hexanol, cyclohexanol, ethylene glycol, diethylene glycol and glycerin; halogenated hydrocarbons such as methylene chloride, 1,1-dichloroethane, 1,2-dichloroethylene, 1-chloropropane, 1-chlorobutane, 1-chloropentane, chlorobenzene, bromobenzene, o-dichlorobenzene and m-dichlorobenzene; amides such as N,N-dimethylformamide, N,N-diethylformamide, N,N-dimethylacetamide, N,N-diethylacetamide and N,N-dimethylmethoxyacetamide; pyrrolidones such as N-methyl-2-pyrrolidone and N-acetyl-2-pyrrolidone; lactones such as γ-butyrolactone and α-acetyl-γ-butyrolactone; sulfoxides such as dimethylsulfoxide; sulfones such as dimethyl sulfone, tetramethylene sulfone and dimethyltetramethylene sulfone; amide phosphates such as hexamethylphosphoramide; and other organic polar solvents. In addition, there may be mentioned aromatic hydrocarbons such as benzene, toluene, xylene and pyridine, and other organic nonpolar solvents. These solvents are used solely or in combination.

Among them, preferred are polar solvents such as propylene glycol monomethyl ether, methyl ethyl ketone, cyclopentanone, cyclohexanone, ethyl acetate, propylene glycol monomethyl ether acetate, N,N-dimethylacetamide, N-methyl-2-pyrrolidone and γ-butyrolactone; aromatic hydrocarbons such as toluene; and mixed solvents thereof.

As the photocurable component, a compound having one or two or more ethylenically unsaturated bond can be used. Examples thereof include amide monomers, (meth)acrylate monomers, urethane (meth)acrylate oligomers, polyester (meth)acrylate oligomers and epoxy (meth)acrylates, hydroxyl group-containing (meth)acrylates and aromatic vinyl compounds such as such as styrene. In the case where the polyimide precursor has a carboxylic acid component (e.g., polyamic acid) in a structure thereof, the use of an ethylenically unsaturated bond-containing compound having a tertiary amino group allows formation of an ionic bond between the tertiary amino group and the carboxylic acid of the polyimide precursor. Therefore, there is an increase in the dissolution rate contrast between the exposed and unexposed regions.

In the case of using such a photocurable composition having an ethylenically unsaturated bond, a photoradical generator may be added further. Examples of the photoradical generator include benzoins and alkyl ethers thereof, such as benzoin, benzoin methyl ether, benzoin ethyl ether and benzoin isopropyl ether; acetophenones such as acetophenone, 2,2-dimethoxy-2-phenylacetophenone, 2,2-diethoxy-2-phenylacetophenone, 1,1-dichloroacetophenone, 1-hydroxyacetophenone, 1-hydroxycyclohexyl phenyl ketone, and 2-methyl-1-[4-(methylthio)phenyl]-2-morpholino-propane-1-on; anthraquinones such as 2-methylanthraquinone, 2-ethylanthraquinone, 2-t-butylanthraquinone, 1-chloroanthraquinone and 2-amylanthraquinone; thioxanthones such as 2,4-dimethylthioxanthone, 2,4-diethylthioxanthone, 2-chlorothioxanthone and 2,4-diisopylthioxanthone; ketals such as acetophenone dimethyl ketal and benzil dimethyl ketal; monoacyl phosphine oxides such as 2,4,6-trimethylbenzoyl diphenylphosphine oxide and bisacyl phosphine oxides; benzophenones such as benzophenone; and xanthones.

To the extent that does not inhibit the advantageous effects of the present invention, other photosensitive component may be added to the photosensitive resin composition of the present invention, the component playing a supplementary role to the base generator of the present invention and generating an acid or base by exposure to light. Also, a base amplifier and/or sensitizer may be added thereto.

Examples of the compound which generates an acid by exposure to light include photosensitive diazoquinone compounds having a 1,2-benzoquinonediazide or 1,2-naphthoquinonediazide structure. Such compounds are described in the specifications of U.S. Pat. Nos. 2,772,972, 2,797,213 and 3,669,658. Also, a conventionally known photobase generator can be used, such as triazine and derivatives thereof, an oxime sulfonate compound, an iodonium sulfonate and a sulfonium sulfonate. Examples of the compound which generates a base by exposure to light include 2,6-dimethyl-3,5-dicyano-4-(2′-nitrophenyl)-1,4-dihydropyridine, 2,6-dimethyl-3,5-diacetyl-4-(2′-nitrophenyl)-1,4-dihydropyridine, and 2,6-dimethyl-3,5-diacetyl-4-(2′,4′-dinitrophenyl)-1,4-dihydropyridine.

A base amplifier may be used in combination, which is decomposed or causes a rearrangement reaction by the action of a small amount of base generated from the base generator, thereby generating a base. Examples of the base amplifier include a compound having a 9-fluorenylmethyl carbamate bond, a compound having a 1,1-dimethyl-2-cyanomethyl carbamate bond ((CN)CH₂C(CH₃)₂OC(O)NR₂) a compound having a p-nitrobenzyl carbamate bond, and a compound having a 2,4-dichlorobenzyl carbamate bond, and urethane-based compounds described in paragraphs [0010] to [0032] of Japanese Patent Application Laid-Open (JP-A) No. 2000-330270 and paragraphs [0033] to [0060] of JP-A No. 2008-250111, for example.

Addition of a sensitizer can be effective when it is required to increase the sensitivity of the photosensitive resin composition by allowing the base generator to sufficiently utilize the energy of electromagnetic waves at a wavelength that passes through the polymer.

Especially in the case where the polyimide precursor has absorption at a wavelength of 360 nm or more, addition of a sensitizer is particularly effective.

Specific examples of compounds called sensitizers include thioxanthone and derivatives thereof such as diethylthioxanthone, coumarins and derivative thereof, ketocoumarin and derivatives thereof, ketobiscoumarin and derivatives thereof, cyclopentanone and derivatives thereof, cyclohexanone and derivatives thereof, thiopyrylium salts and derivatives thereof, thioxanthenes and derivatives thereof, and xanthenes and derivatives thereof.

Specific examples of coumarins, ketocoumarin and derivatives thereof include 3,3′-carbonylbiscoumarin, 3,3′-carbonylbis(5,7-dimethoxycoumarin) and 3,3′-carbonylbis(7-acetoxycoumarin). Specific examples of thioxanthone and derivatives thereof include diethylthioxanthone and isopropylthioxanthone. In addition, there may be mentioned benzophenone, acetophenone, phenanthrene, 2-nitrofluorene, 5-nitroacenaphthene, benzoquinone, 2-ethylanthraquinone, 2-tert-butylanthraquinone, 1,2-benzanthraquinone, 1,2-naphthoquinone, etc.

They exert particularly excellent effects when combined with a base generator, so that a sensitizer which exerts optimal sensitizing effects is appropriately selected depending on the structure of the base generator.

Other various kinds of organic or inorganic, low- or high-molecular-weight compounds may be added further to provide processability and various kinds of functionality to the resin composition of the present invention. For example, there may be used a dye, a surfactant, a leveling agent, a plasticizer, fine particles, etc. Examples of the fine particles include organic fine particles such as polystyrene and polytetrafluoroethylene, and inorganic fine particles such as colloidal silica, carbon and phyllosilicate. They may be porous or hollow. Functions or forms thereof include a pigment, filler, fiber, etc.

From the viewpoint of film physical properties of the resulting pattern, especially film strength and heat resistance, the polymer precursor (solid content) contained in the photosensitive resin composition of the present invention is generally 0.1 to 99.9% by weight, preferably 0.5 to 70% by weight of the total solid content of the photosensitive resin composition. In the present invention, “solid content” means all components other than the solvent and includes a monomer which is liquid at room temperature.

The base generator of the present invention is generally contained in the range of 0.1 to 80% by weight, preferably in the range of 0.1 to 60% by weight of the total solid content of the photosensitive resin composition. If less than 0.1% by weight, the dissolution contrast between the exposed and unexposed regions could not be increased sufficiently. If more than 80% by weight, properties of the finally-obtained cured resin are poorly reflected in the final product.

In the case of using the base generator of the present invention as a curing agent, the base generator is generally contained in the range of 0.1 to 80% by weight, preferably in the range of 0.5 to 60% by weight of the total solid content of the photosensitive resin composition, depending of the degree of curing.

In the case of using the base generator of the present invention as a curing accelerator, the photosensitive resin composition can be cured by adding the base generator in a small amount. The base generator of the present invention is generally contained in the range of 0.1 to 30% by weight, preferably in the range of 0.5 to 20% by weight of the total solid content of the photosensitive resin composition.

In the photosensitive resin composition of the present invention, the polymer precursor (solid content) is generally 50.1 to 99.9% by weight, preferably 62.5 to 99.5% by weight of the total solid content of the photosensitive resin composition. The base generator represented by the chemical formula (1) is generally 0.1 to 49.9% by weight, preferably 0.5 to 37.5% by weight of the total solid content of the photosensitive resin composition.

The solid content of the photosensitive resin composition refers to all components other than a solvent and includes a liquid monomer component.

A mixing ratio of other optional component(s) other than a solvent is preferably in the range of 0.1% to 95% by weight of the total solid content of the photosensitive resin composition. If less than 0.1% by weight, addition of the additive(s) is not effective very much. If more than 95% by weight, properties of the finally-obtained cured resin are poorly reflected in the final product.

The photosensitive resin composition of the present invention can be used in various kinds of coating and molding processes and can produce films and three-dimensional molded products.

As described above, according to the present invention, the photosensitive resin composition can be obtained by such a simple method of mixing the polymer precursor with the base generator of the present invention; therefore, the present invention provides excellent cost performance.

An aromatic component-containing carboxylic acid and basic substance which constitute the base generator of the present invention are available at low cost; therefore, the price of the photosensitive resin composition can be low.

Due to the base generator of the present invention, the photosensitive resin composition of the present invention can be used to promote the reaction of various kinds of polymer precursors into a final product, and the structure of the finally-obtained polymer can be selected from a wide range of structures.

Moreover, the base generator of the present invention is cyclized when producing a base and loses a phenolic hydroxyl group. Therefore, the solubility of the base generator in a developer such as a basic solution is changed and when the polymer precursor is a polyimide precursor, polybenzoxazole precursor or the like, the base generator supports the solubility decrease of the photosensitive resin composition and contributes to increasing the dissolution contrast between the exposed and unexposed regions.

Also, due to the catalytic effect of the basic substance generated by exposure to electromagnetic radiation, such as amine, it is possible to decrease a process temperature that is required for a reaction such as cyclization such as imidization of the polyimide precursor or polybenzoxazole precursor into a final product. Therefore, it is possible to reduce the load on the process and heat damage to a final product.

In addition, when a heating step is included in the process of obtaining a final product from the polymer precursor, the heating step can be utilized by the base generator of the present invention which generates a base by exposure to electromagnetic radiation and heating; therefore, it is possible to reduce the amount of exposure to electromagnetic radiation and to use the step efficiently.

The photosensitive resin composition of the present invention can be used in all conventionally-known fields and products which use a resin material, such as a printing ink, a paint, a sealing agent, an adhesive, an electronic material, an optical circuit component, a molding material, a resist material, a building material, a stereolithography product and an optical element. It can be suitably used in any of applications such as an application in which the photosensitive resin composition is subjected to whole surface exposure, such as a paint, a sealing agent and an adhesive, and an application in which the photosensitive resin composition is used to form a pattern, such as a permanent film and a stripping film.

The photosensitive resin composition of the present invention is suitably used in a wide range of fields and products for which properties such as heat resistance, dimensional stability and insulation are effective, such as a paint, a printing ink, a sealing agent, an adhesive or a material for forming displays, semiconductor devices, electronic components, microelectromechanical systems (MEMS), optical elements or building materials. For example, in particular, as the material for forming electronic components, the photosensitive resin composition can be used for a printed wiring board, an interlayer insulating film, a wire cover film or the like as an encapsulating material or layer forming material. As the material for forming displays, the photosensitive resin composition can be used for a color filter, a film for flexible displays, a resist material, an orientation film or the like as a layer forming material or image forming material. As the material for forming semiconductor devices, it can be used as a resist material, a material for forming layers such as a buffer coat film, etc. As the material for forming optical components, it can be used for a hologram, an optical waveguide, an optical circuit, an optical circuit component, an antireflection film or the like as an optical material or layer forming material. As the building material, it can be used for a paint, a coating agent or the like. Also, it can be used as the material for stereolithography products. The photosensitive resin composition of the present invention provides any of the following articles: a paint, a sealing agent, an adhesive, a display, a semiconductor device, an electronic component, a microelectromechanical system, a stereolithography product, an optical element and a building material.

Because of having the above characteristics, the photosensitive resin composition of the present invention can be also used as a pattern forming material. Especially in the case where the photosensitive resin composition containing the polyimide precursor or polybenzoxazole precursor is used as a pattern forming material (resist), the pattern formed therewith is a permanent film that comprises polyimide or polybenzoxazole and functions as a component which provides heat resistance or insulation property. For example, it is suitable to form a color filter, a film for flexible displays, an electronic component, a semiconductor device, an interlayer insulating film, a wire cover film, an optical circuit, an optical circuit component, an antireflection film, other optical element or an electronic member.

The present invention also provides an article selected from a printed product, a paint, a sealing agent, an adhesive, a display device, a semiconductor device, an electronic component, a microelectromechanical system, a stereolithography product, an optical element or a building material, wherein at least part of each of which articles comprises the photosensitive resin composition of the present invention or a cured product thereof.

### <Pattern Forming Method>

The pattern forming method of the present invention is a method for forming a pattern by forming a coating film or molded body with the photosensitive resin composition of the present invention, exposing the coating film or molded body to electromagnetic radiation in a predetermined pattern, heating the coating film or molded body after or at the same time as the exposure to change the solubility of the exposed region, and then developing the coating film or molded body.

A coating film is formed by applying the photosensitive resin composition of the present invention onto a substrate of some sort, or a molded body is formed by an appropriate molding method using the photosensitive resin composition. The coating film or molded body is exposed to electromagnetic radiation in a predetermined pattern and heated after or at the same time as the exposure, so that the base generator of the present invention is isomerized and cyclized only in the exposed region, thereby generating a basic substance. The basic substance functions as a catalyst that promotes the reaction of the polymer precursor in the exposed region into a final product.

In the case of using a polymer precursor of which thermal curing temperature can be decreased by the catalytic reaction of a base, such as a polyimide precursor and polybenzoxazole precursor, a region where a pattern is required to be left on the coating film or molded body formed with the photosensitive resin composition is exposed first, the photosensitive resin composition comprising a combination of such a polymer precursor and the base generator of the present invention. By heating the same after or at the same time as the exposure, a basic substance is generated in the exposed region and the thermal curing temperature of the region is selectively decreased. After or at the same time as the exposure, the coating film or molded body is heated at a treatment temperature at which the exposed region is thermally cured while the unexposed region is not, thereby curing only the exposed region. The heating process for generating a basic substance and another heating process for causing a reaction to cure the exposed region only (post exposure bake) may be one single process or different processes. Next, the unexposed region is dissolved with a predetermined developer (such as an organic solvent or basic aqueous solution) to form a pattern comprising a thermally-cured product. This pattern is heated further as needed to finish thermal curing. A desired two-dimensional resin pattern (general plane pattern) or three-dimensional resin pattern (three-dimensionally formed pattern) is obtained by these processes, both of which are normally negative patterns.

Even in the case of using a polymer precursor that can initiate a reaction by the catalytic action of a base, such as a compound or polymer having an epoxy or cyanate group, the region where a pattern is required to be left on the coating film or molded body formed with the photosensitive resin composition is exposed first, the photosensitive resin composition comprising a combination of such a polymer precursor and the base generator of the present invention. By heating the same after or at the same time as the exposure, a basic substance is generated in the exposed region and thus the compound or polymer having an epoxy or cyanate group in the region initiates a reaction to cure only the exposed region. The heating process for generating a basic substance and another heating process for causing a reaction to cure the exposed region only (post exposure bake) may be one single process or different processes. Next, the unexposed region is dissolved with a predetermined developer (such as an organic solvent or basic aqueous solution) to form a pattern comprising a thermally-cured product. This pattern is heated further as needed to finish thermal curing. A desired two-dimensional resin pattern (general plane pattern) or three-dimensional resin pattern (three-dimensionally formed pattern) is obtained by these processes, both of which are normally negative patterns.

The photosensitive resin composition of the present invention forms a non-adhesive coating film on a substrate by: dissolving the same in a polar solvent such as propylene glycol monomethyl ether, methyl ethyl ketone, cyclopentanone, cyclohexanone, ethyl acetate, propylene glycol monomethyl ether acetate, N,N-dimethylacetamide, N-methyl-2-pyrrolidone or γ-butyrolactone, an aromatic hydrocarbon such as toluene, or a mixed solvent thereof; applying the mixture onto a surface of a substrate such as a silicon wafer, metal substrate, ceramic substrate or resin film by a dipping method, spraying method, flexographic printing method, gravure printing method, screen printing method, spin coating method, dispensing method or the like; and heating the applied coating film to remove most of the solvent, thereby forming the non-adhesive film on the substrate. A thickness of the coating film is not particularly limited and is preferably 0.5 to 50 μm. From the viewpoint of sensitivity and development rate, it is more preferably 1.0 to 20 μm. A drying condition of the applied coating film is a temperature of 80 to 100° C. and a time of 1 to 20 minutes, for example.

The coating film is exposed to electromagnetic radiation through a mask having a predetermined pattern so as to be exposed in a predetermined pattern. After heating, the film is developed with an appropriate developer to remove the unexposed region of the film, thereby obtaining a desirably patterned film.

An exposing method and device used in the exposure process are not particularly limited. The method may be contact exposure or indirect exposure. As the device, there may be used a contact-proximity exposure system using a g-line stepper, i-line stepper or super high pressure mercury lamp, a mirror projection exposure system, or other projection device or radiation source which can emit ultraviolet light, visible light, X-ray, electron beam or the like.

The heating temperature for generating a base after or at the same time as the exposure is appropriately determined depending on the polymer precursor to be combined or on the intended purpose, and it is not particularly limited. The heating may be heating at a temperature of the environment where the photosensitive resin composition is placed (e.g., room temperature) and in this case, bases are gradually generated. Bases are also generated by heat that is produced as a by-product of the exposure to electromagnetic radiation, so that heating may be substantially performed at the same time by the heat produced as the by-product. To increase the reaction rate and efficiently generate an amine, the heating temperature for generating a base is preferably 30° C. or more, more preferably 60° C. or more, still more preferably 100° C. or more, and particularly preferably 120° C. or more. However, the suitable heating temperature is not limited thereto because the unexposed region can be cured by heating at 60° C. or more for example, depending on the type of the polymer precursor used in combination.

For example, in the case of an epoxy resin, the preferred temperature range of heat treatment is appropriately determined depending on the type of the epoxy resin; however, it is generally about 100° C. to 150° C.

To physically promote a crosslinking reaction or initiate a reaction for curing only the exposed region, it is preferable to perform a post exposure bake (PEB) on the coating film of the photosensitive resin composition of the present invention between the exposure and developing processes. The PEB is preferably performed at a temperature at which, due to the action of the base generated by the exposure to electromagnetic radiation and heating, the reaction rate of a curing reaction (e.g., imidization rate) will be different between the exposed region where the base is present and the unexposed region where the base is not present. For example, in the case of imidization, the preferred temperature range of heat treatment is generally about 60° C. to 200° C., more preferably 120° C. to 200° C. When the heat treatment temperature is less than 60° C., imidization is not efficient and it is difficult to cause a difference between the imidization rate of the exposed region and that of the unexposed region under a realistic process condition. When the heat treatment temperature exceeds 200° C., imidization could proceed even in the unexposed region where no amine is present, so that it is difficult to cause a difference between the solubility of the exposed region and that of the unexposed region.

The heat treatment may be performed by any conventionally method. A specific example thereof is, but not particularly limited to, heating with a circulation-type oven or hot plate in the air or a nitrogen atmosphere.

In the present invention, a base is generated from the base generator by exposure to electromagnetic radiation and heating; however, the heating for generating a base and PEB process may be one single process or different processes.

### (Developer)

The developer used in the developing process is not particularly limited as long as it is a solvent which can change the solubility of the exposed region. It can be appropriately selected from basic aqueous solutions, organic solvents and so on, depending on the used polymer precursor.

The basic aqueous solutions are not particularly limited and include, for example, a tetramethylammonium hydroxide (TMAH) aqueous solution in a concentration of 0.01% by weight to 10% by weight (preferably 0.05% by weight to 5% by weight) and aqueous solutions of diethanolamine, diethyl amino ethanol, sodium hydroxide, potassium hydroxide, sodium carbonate, potassium carbonate, sodium hydrogencarbonate, potassium hydrogencarbonate, triethylamine, diethylamine, methylamine, dimethylamine, dimethylamino ethyl acetate, dimethylaminoethanol, dimethylamino ethyl methacrylate, cyclohexylamine, ethylenediamine, hexamethylenediamine, tetramethylammonium and so on.

A solute may be one kind or two or more kinds. The basic aqueous solution may contain an organic solvent or the like when it contains water in an amount of 50% or more, more preferably 70% or more of the total weight thereof.

The organic solvent is not particularly limited. As the organic solvent, polar solvents such as N-methyl-2-pyrrolidone, N,N-dimethylformamide, N,N-dimethylacetamide, dimethylsulfoxide, γ-butyrolactone and dimethylacrylamide, alcohols such as methanol, ethanol and isopropanol, esters such as ethyl acetate and propylene glycol monomethyl ether acetate, ketones such as cyclopentanone, cyclohexanone, isobutyl ketone and methyl isobutyl ketone, and other organic solvents such as tetrahydrofuran, chloroform and acetonitrile, may be used solely or in combination of two or more kinds. After the development, washing is performed with water or a poor solvent. Even in this case, an alcohol such as ethanol or isopropyl alcohol, an ester such as ethyl lactate or propylene glycol monomethyl ether acetate, etc., may be added to water.

After the development, to stabilize the pattern, rinsing with water or a poor solvent is performed as needed and then drying is performed at a temperature of 80 to 100° C. To make the resulting relief pattern heat resistant, it is heated at a temperature of 180 to 500° C., more preferably 200 to 350° C. for several ten minutes to several hours, thereby forming a patterned, highly heat-resistant resin layer.

### EXAMPLES

Hereinafter, the present invention will be described in detail by way of examples. The scope of the present invention is not restricted by these examples. All designations of “part” or “parts” are part or parts by weight unless otherwise specifically indicated. Chemical structures of base generators generated in the following examples were confirmed by ¹H NMR measurement.

Measurements and experiments were carried out by means of the following devices:

¹H NMR measurement: JEOL JNM-LA400WB manufactured by JEOL Ltd.

Manual exposure: MA-1100 manufactured by Japan Science Engineering Co., Ltd.

Measurement of absorbance: Ultraviolet-visible spectrophotometer UV-2550 manufactured by Shimadzu Corporation

Measurement of 5% weight loss temperature:

Thermogravimetric/differential thermal analyzer DTG-60 manufactured by Shimadzu Corporation

Measurement of infrared absorption spectra: FTS 7000 manufactured by Varian Technologies Japan Ltd.

Heating of coating film: HOT PLATE EC-1200 manufactured by AS ONE Corporation (It may be referred to as “hot plate” in the following examples)

### Synthesis Example 1

### Synthesis of Polyimide Precursor

Di(4-aminophenyl)ether of 10.0 g (50 mmol) was poured into a 300 mL three-neck flask, dissolved in 105.4 mL of dehydrated N,N-dimethylacetamide (DMAc) and stirred while cooling in an ice bath under a nitrogen flow. 3,3′,4,4′-biphenyltetracarboxylic acid-3, 4:3′,4′-dianhydride of 14.7 g (50 mmol) was gradually added thereto and stirred in an ice bath for five hours after the addition. The resulting solution was reprecipitated with dehydrated diethyl ether and the resulting precipitate was dried for 17 hours at a room temperature under a reduced pressure, thereby obtaining a polyamic acid having a weight average molecular weight of 10,000 (polyimide precursor (1)) quantitatively in the form of a white solid.

### Synthesis Example 2

### Synthesis of Metal Alkoxide Condensate

Into a 100 ml flask provided with a condenser tube, phenyltriethoxysilane of 5 g, triethoxysilane of 10 g, ammonia water of 0.05 g, water of 5 ml and propylene glycol monomethyl ether acetate of 50 ml were poured. The resulting solution was stirred with a semicircular-type mechanical stirrer and reacted with a heating mantle for six hours at 70° C. Then, ethanol produced by a condensation reaction with water, and residual water were removed with an evaporator. After the reaction was completed, the flask was left to reach room temperature, thereby producing a condensate of alkoxysilane (alkoxysilane condensate (1)).

### Production Example 1

### Synthesis of Base Generator (1)

In a 100 mL flask, potassium carbonate of 1.00 g was added to methanol of 10 mL. In a 50 mL flask, ethoxycarbonylmethyl (triphenyl)phosphonium bromide (manufactured by Tokyo Chemical Industry Co., Ltd.) of 4.29 g (7.76 mmol) and bis(3-formyl-4-hydroxyphenyl)methane (manufactured by Asahi Organic Chemicals Industry Co., Ltd.) of 1.00 g (3.88 mmol) were dissolved in tetrahydrofuran of 10 mL and gradually added dropwise to the potassium carbonate solution stirred well. After the mixture was stirred for three hours, reaction completion was confirmed by thin-layer chromatography, and then the potassium carbonate was removed by filtration. The resultant was subjected to vacuum concentration. After the concentration, a 1 N sodium hydroxide aqueous solution of 15 mL was added thereto and stirred overnight. After reaction completion, precipitates were removed by filtration and concentrated hydrochloric acid was added dropwise to the resulting reaction solution to acidulate the solution. The thus-obtained precipitate was collected by filtration and washed with a small amount of chloroform, thereby obtaining acid derivative A.

In a 100 mL three-neck flask under a nitrogen atmosphere, acid derivative A of 300 mg (880 μmol) was dissolved in dehydrated tetrahydrofuran of 20 mL, and 1-ethyl-3-(3-dimethylaminopropyl) carbodiimide hydrochloride (manufactured by Tokyo Chemical Industry Co., Ltd.) of 405 mg (5.2 mmol) was added thereto in an ice bath. Thirty minutes later, 1-hydroxybenzotriazole (manufactured by Tokyo Chemical Industry Co., Ltd.) of 0.79 g (2.1 mmol) was gradually added dropwise. After the mixture was stirred for about 30 minutes, piperidine (manufactured by Kanto Chemical Co., Inc.) of 0.21 ml (2.1 mmol) was added thereto and then the mixture was stirred overnight. After reaction completion, the resulting reaction solution was condensed and dissolved in water. After extraction with chloroform, the resultant was washed with a hydrogen carbonate aqueous solution, 1 N hydrochloric acid and then saturated saline, and dried with sodium sulfate. The resultant was purified by silica-gel column chromatography (developing solvent: chloroform/methanol=100/1 to 10/1), thereby obtaining base generator (1) represented by the following chemical formula (8) of 100 mg.

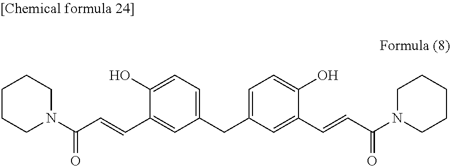

### Production Example 2

### Synthesis of Base Generator (2)

In a 500 mL recovery flask, 4,4′-dihydroxydiphenyl ether (manufactured by Tokyo Chemical Industry Co., Ltd.) of 14.6 g (72.4 mmol) and hexamethylenetetramine (manufactured by Tokyo Chemical Industry Co., Ltd.) of 15.2 g (109 mmol, 1.5 eq) were dissolved in trifluoroacetic acid (manufactured by Kanto Chemical Co., Inc.) of 100 ml and reacted at 95° C. for 10 hours. After the reaction was completed, in an ice bath, 1 N hydrochloric acid of 200 ml was added thereto and the resultant was stirred for 15 minutes. After the stirring was completed, the resultant was extracted with chloroform to obtain an extract. The extract was washed with hydrochloric acid and saturated saline, thereby obtaining 5,5′-oxybis(2-hydroxybenzaldehyde) of 1.27 g.

Acid derivative B was obtained in the same manner as Production example 1, except that an equimolar amount of 5,5′-oxybis(2-hydroxybenzaldehyde) was used in place of bis(3-formyl-4-hydroxyphenyl)methane. Then, an amidation reaction was performed in the same manner as Production example 1, except that an equimolar amount of acid derivative B was used in place of acid derivative A. The resultant was purified by silica-gel column chromatography (developing solvent: chloroform/methanol 100/1 to 50/1), thereby obtaining base generator (2) represented by the following chemical formula (9).

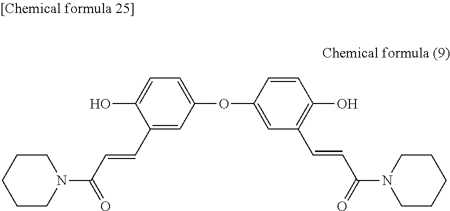

### Production Example 3

### Synthesis of Base Generator (3)

5,5′-methylenebis(2-hydroxy-3-methylbenzaldehyde) was obtained in the same manner as Production example 2, except that an equimolar amount of 4,4′-methylenebis(2-methylphenol) (manufactured by Tokyo Chemical Industry Co., Ltd.) was used in place of 4,4′-dihydroxydiphenyl ether.

Next, acid derivative C was obtained in the same manner as Production example 1, except that an equimolar amount of 5,5′-methylenebis(2-hydroxy-3-methylbenzaldehyde) was used in place of bis(3-formyl-4-hydroxyphenyl)methane. Then, an amidation reaction was performed in the same manner as Production example 1, except that an equimolar amount of acid derivative C was used in place of acid derivative A. The resultant was purified by silica-gel column chromatography (developing solvent: chloroform/methanol 100/1 to 50/1), thereby obtaining base generator (3) represented by the following chemical formula (10).

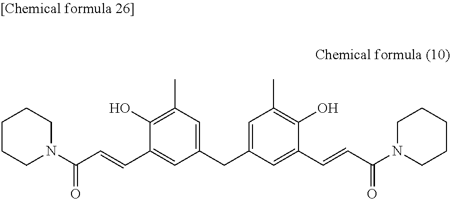

### Production Example 4

### Synthesis of Base Generator (4)

2,5-dihydroxy-1,4-benzenedicarboxaldehyde was obtained with reference to the method described on pages 417 to 419 of Journal of Heterocyclic Chemistry (1975), 12(2).

Next, acid derivative D was obtained in the same manner as Production example 1, except that an equimolar amount of 2,5-dihydroxy-1,4-benzenedicarboxaldehyde was used in place of bis(3-formyl-4-hydroxyphenyl)methane. Then, an amidation reaction was performed in the same manner as Production example 1, except that an equimolar amount of acid derivative D was used in place of acid derivative A. The resultant was purified by silica-gel column chromatography (developing solvent: chloroform/methanol 100/1 to 50/1), thereby obtaining base generator (4) represented by the following chemical formula (11).

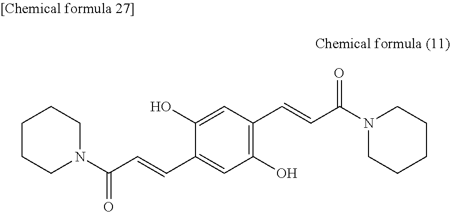

### Production Example 5

### Synthesis of Base Generator (5)

3,7-dihydroxy-9,10-dioxo-9,10-dihydroanthracene-2,6-dicarbaldehyde) was obtained in the same manner as Production example 2, except that an equimolar amount of anthraflavic acid (manufactured by Tokyo Chemical Industry Co., Ltd.) was used in place of 4,4′-dihydroxydiphenyl ether.

Next, acid derivative E was obtained in the same manner as Production example 1, except that an equimolar amount of 3,7-dihydroxy-9,10-dioxo-9,10-dihydroanthracene-2,6-dicarbaldehyde was used in place of bis(3-formyl-4-hydroxyphenyl)methane. Then, an amidation reaction was performed in the same manner as Production example 1, except that an equimolar amount of acid derivative E was used in place of acid derivative A. The resultant was purified by silica-gel column chromatography (developing solvent: chloroform/methanol 100/1 to 50/1), thereby obtaining base generator (5) represented by the following chemical formula (5).

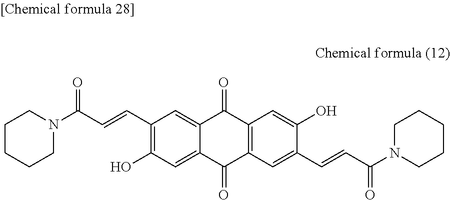

### Production Example 6

### Synthesis of Base Generator (6)

In a 100 mL three-neck flask, 2,4-dihydroxy-cinnamic acid (manufactured by Aldrich Corp.) of 2.0 g (11.1 mmol) was dissolved in tetrahydrofuran of 10 mL. 1-ethyl-3-(3-dimethylaminopropyl)carbodiimide hydrochloride (EDC) (manufactured by Tokyo Chemical Industry Co., Ltd.) of 2.56 g (13.3 mmol, 1.2 eq) was added thereto. Thirty minutes later, piperidine (manufactured by Tokyo Chemical Industry Co., Ltd.) of 1.28 mL (13.3 mmol) was added thereto. After reaction completion, the resultant was dissolved in water and extracted with chloroform to obtain an extract. The extract was washed with a saturated sodium hydrogen carbonate aqueous solution, 1 N hydrochloric acid and saturated saline. Then, the resultant was purified by silica-gel column chromatography (developing solvent: chloroform/methanol 100/1 to 10/1 (by volume ratio)), thereby obtaining (E)-3-(2,4-dihydroxyphenyl)-1-(piperidin-1-yl)prop-2-en-1-one of 1.42 g.

Then, in a 100 ml flask under an argon atmosphere, (E)-3-(2,4-dihydroxyphenyl)-1-(piperidin-1-yl)prop-2-en-1-one) of 1.0 g (4.04 mmol) and epichlorohydrin (manufactured by Tokyo Chemical Industry Co., Ltd.) of 0.80 ml (10.1 mmol) were dissolved in methanol of 10 ml and refluxed. Potassium hydroxide (manufactured by Kanto Chemical Co., Inc.) of 0.24 g (4.44 mmol) was dissolved in methanol of 1.0 ml and gradually added dropwise thereto. After stirring the mixture for three hours, the mixture was returned to room temperature and filtered. The filtrate was condensed, dissolved in dichloromethane and washed with water. Then, the resultant was purified by silica-gel column chromatography (developing solvent: chloroform/methanol=100/1 to 10/1), thereby obtaining (E)-3-(2-hydroxy-4-(oxiran-2-ylmethoxy)phenyl)-1-(piperidin-1-yl)prop-2-en-1-one of 620 mg.

In a 10 ml flask, polyacrylic acid (weight average molecular weight: 1,800) (manufactured by Aldrich Corp.) of 50 mg was dissolved in dimethylformamide of 2 ml. After the reaction solution was heated to 110° C., (E)-3-(2-hydroxy-4-(oxirane-2-ylmethoxy)phenyl)-1-(piperidin-1-yl)prop-2-en-1-one of 230 mg (760 μmol) was added thereto and stirred for six hours. The reaction solution was returned to room temperature and added to hexane of 10 ml. The resultant was filtered, thereby obtaining base generator (6) which is a polymer having a repeating unit represented by the following chemical formula (13). The polymer was found to have a weight average molecular weight of 9,500 by GPC measurement.

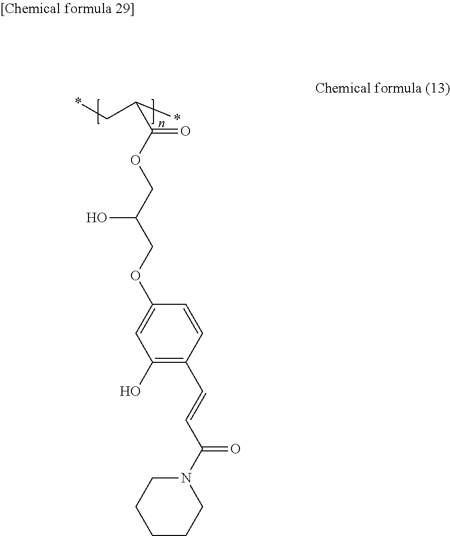

### Production Example 7

### Synthesis of Base Generator (7)

(E)-3-(2 hydroxy-4-(oxirane-2-ylmethoxy)phenyl)-1-(piperidin-1-yl)prop-2-en-1-one was obtained in the same manner as Production example 6.

In a 10 ml flask, polyacrylic acid (weight average molecular weight: 1,800) (manufactured by Aldrich Corp.) of 50 mg was dissolved in dimethylformamide of 2 ml. After the reaction solution was heated to 110° C., (E)-3-(2 hydroxy-4-(oxirane-2-ylmethoxy)phenyl)-1-(piperidin-1-yl)prop-2-en-1-one 115 mg (380 μmol) was added thereto and stirred for six hours. The reaction solution was returned to room temperature and added to hexane of 10 ml. The resultant was filtered, thereby obtaining base generator (7) which is a polymer having a repeating unit represented by the following chemical formula (14) wherein n:m=5:4. The polymer was found to have a weight average molecular weight of 6,100 by GPC measurement.

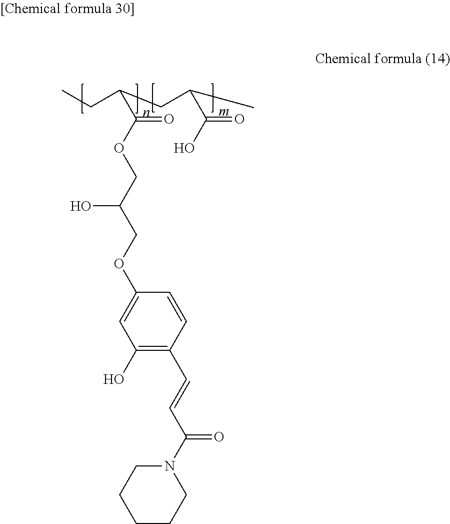

### Production Example 8

### Synthesis of Base Generator (8)

(E)-3-(2-hydroxy-4-(oxirane-2-ylmethoxy)phenyl)-1-(piperidin-1-yl)prop-2-en-1-one was obtained in the same manner as Production example 6.

Next, in a 100 ml flask, (E)-3-(2-hydroxy-4-(oxirane-2-ylmethoxy)phenyl)-1-(piperidin-1-yl)prop-2-en-1-one of 0.2 g (660 μmol) was dissolved in dimethylformamide of 5 ml. After the reaction solution was heated to 110° C. while performing air bubbling, p-methoxyphenol (manufactured by Tokyo Chemical Industry Co., Ltd.) of 0.8 mg (6.6 μmol) and acrylic acid (manufactured by Tokyo Chemical Industry Co., Ltd.) of 49.8 μl (730 μmol) were added thereto and stirred for six hours. After the reaction solution was returned to room temperature, it was dissolved in ethyl acetate and washed with a saturated sodium hydrogen carbonate aqueous solution and 1 N hydrochloric acid. Then, the resultant was dried with magnesium sulfate and condensed. The resultant was purified by silica-gel column chromatography (developing solvent: chloroform/methanol=100/1 to 10/1), thereby obtaining (E)-2-hydroxy-3-(3-hydroxy-4-(3-oxo-3-(piperidin-1-yl)prop-1-enyl)phenoxy)propyl acrylate of 0.21 g.

In a 10 ml flask under a nitrogen atmosphere, (E)-2-hydroxy-3-(3-hydroxy-4-(3-oxo-3-(piperidin-1-yl)prop-1-enyl)phenoxy)propyl acrylate of 50 mg was dissolved in dimethylformamide of 2 ml. After the reaction solution was heated to 85° C., 2,2′-azobis(isobutyronitrile) (manufactured by Tokyo Chemical Industry Co., Ltd.) of 0.5 mg was added thereto and stirred for six hours. The reaction solution was returned to room temperature and added to hexane of 10 ml. The resultant was filtered, thereby obtaining base generator (8) which is a polymer having a repeating unit represented by the following chemical formula (15). The polymer was found to have a weight average molecular weight of 26,300 by GPC measurement.

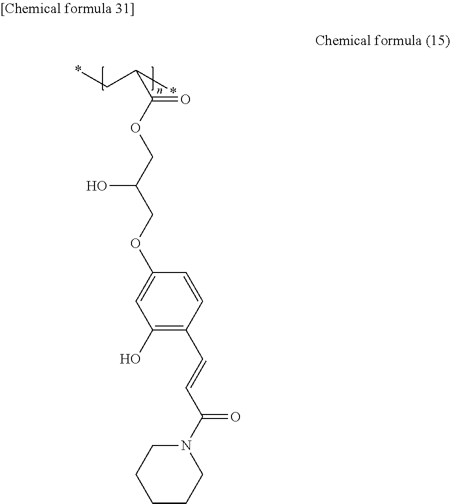

### Production Example 9

### Synthesis of Base Generator (9)

(E)-2-hydroxy-3-(3-hydroxy-4-(3-oxo-3-(piperidin-1-yl)prop-1-enyl)phenoxy)propyl acrylate was obtained in the same manner as Production example 8.

In a 10 ml flask under a nitrogen atmosphere, (E)-2-hydroxy-3-(3-hydroxy-4-(3-oxo-3-(piperidin-1-yl)prop-1-enyl)phenoxy)propyl acrylate of 50 mg and methyl acrylate (manufactured by Tokyo Chemical Industry Co., Ltd.) of 12 μl was dissolved in dimethylformamide of 2 ml. After the reaction solution was heated to 85° C., 2,2′-azobis(isobutyronitrile) (manufactured by Tokyo Chemical Industry Co., Ltd.) of 0.5 mg was added thereto and stirred for six hours. The reaction solution was returned to room temperature and added to hexane of 10 ml. The resultant was filtered, thereby obtaining base generator (9) which is a polymer having a repeating unit represented by the following chemical formula (16) wherein n:m=7:5. The polymer was found to have a weight average molecular weight of 36,500 by GPC measurement.

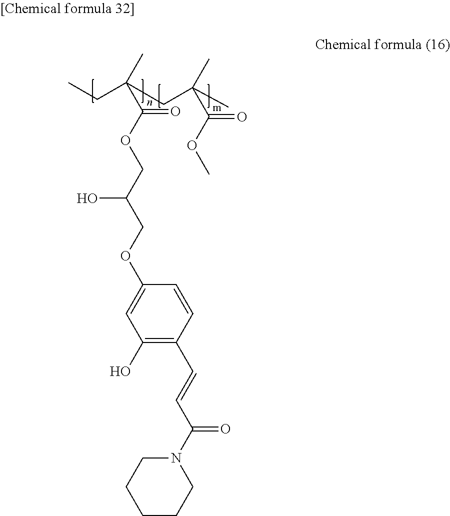

### Comparative Production Example 1

### Synthesis of Comparative Base Generator (1)

A compound represented by the following chemical formula (17) was synthesized as comparative base generator (1), according to the description of Japanese Patent Application Laid-Open (JP-A) No. 2009-80452.

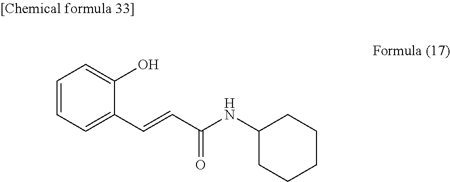

### <Evaluation of Base Generators>

The thus-synthesized base generators (1) to (9) and comparative base generator (1) were subjected to the following measurements for evaluation. Results of molar absorption coefficient measurement and 5% weight loss temperature measurement are shown in Table 1.

### (1) Molar Absorption Coefficient

Each of base generators (1) to (9) and comparative base generator (1) was dissolved in acetonitrile to have a concentration of 1×10⁻⁴ mol/L, and the resulting solution was poured into a quartz cell (optical path 10 mm) to measure the absorbance. Molar absorption coefficient ∈ is a value obtained by dividing the absorbance of a solution by the thickness of an absorbing layer and the molarity of a solute.

### (2) 5% Weight Loss Temperature

To evaluate heat resistance, each of base generators (1) to (9) and comparative base generator(1) was measured for 5% weight loss temperature in the condition of a sample weight of 3.4 mg and a heating rate of 10° C./min.

TABLE 1

| | | | 5% weight |
|---|---|---|---|
| | Molar absorption | | loss |
| | coefficient (ε) | | temperature |
| | 365 nm | 405 nm | (° C.) |
| Base generator (1) | 520 | 110 | 235 |
| Base generator (2) | 4020 | 160 | 220 |
| Base generator (3) | 540 | 120 | 234 |
| Base generator (4) | 7200 | 520 | 190 |
| Base generator (5) | 8320 | 490 | 185 |
| Base generator (6) | 450 | 20 | 189 |
| Base generator (7) | 360 | 30 | 192 |
| Base generator (8) | 420 | 10 | 190 |
| Base generator (9) | 390 | 20 | 188 |
| Comparative base generator (1) | 30 | 0 | 199 |

### (3) Base-Generating Ability

Base-generating ability was evaluated by NMR measurement. “Base generation rate” is the percentage of the molar number of generated bases with respect to the molar number of a base generator used. Each of the base generation rates of base generators (1) to (9) and comparative base generator (1) is the ratio of a combination of exposure to light and heating.

A set of two 1-mg samples were taken from each of base generators (1) to (9). Each of the samples was dissolved in dimethyl-d6 sulfoxide of 0.5 mL in a quartz NMR tube. Among the two samples of each base generator, using a filter that transmits 20% of i-line and a high-pressure mercury lamp, one sample was exposed to light at J/cm², while the other sample was not exposed to light. The samples were measured for ¹H NMR to determine the isomerization rate of each.

In the case of base generator (1), 42.9% of the same was isomerized when exposed to light at 20 J/cm². When the isomerized sample was heated at 160° C. for 10 minutes, 100% of the isomerized compounds were cyclized, thereby generating bases. The base generation rate of comparative base generator (1) was obtained in the same manner, and 33.3% of the comparative base generator was isomerized when exposed to light at 20 J/cm². As a result of heating the isomerized sample at 160° C. for 10 minutes, bases were generated.

It is clear from the above results that base generator (1) of the present invention has a higher sensitivity than comparative base generator (1).

The base-generating ability of base generators (2) to (9) were also evaluated in the same manner. The results are shown in Table 2.

TABLE 2

Electromagnetic radiation
[J/cm²]

| | 0 | 2 | 20 |
|---|---|---|---|
| Base generator (1) | 0 | 6.7 | 42.9 |
| Base generator (2) | 0 | 8.3 | 61.0 |
| Base generator (3) | 0 | 6.9 | 43.1 |
| Base generator (4) | 0 | 32.0 | 86.4 |
| Base generator (5) | 0 | 34.2 | 89.1 |
| Base generator (6) | 0 | 7.2 | 43.5 |
| Base generator (7) | 0 | 7.4 | 46.2 |
| Base generator (8) | 0 | 6.9 | 45.9 |
| Base generator (9) | 0 | 7.1 | 41.3 |
| Comparative base generator (1) | 0 | 5.1 | 33.3 |

### Example 1

### Production of Photosensitive Resin Composition (1)

Photosensitive resin composition (1) having the following composition was produced.

Polyimide precursor (1): 85 parts by weight

Base generator (1): 15 parts by weight

Solvent NMP (N-methylpyrrolidone): 843 parts by weight

### Comparative Example 1

### Production of Comparative Photosensitive Resin Composition (1)

Comparative photosensitive resin composition (1) was produced in the same manner as in Example 1, except that comparative base generator (1) was used in place of base generator (1).

### (Production of Coating Film)

Each of photosensitive resin composition (1) and comparative photosensitive resin composition (1) was spin-coated on a chrome-plated glass plate so as to have a final film thickness of 4 μm and dried on a hot plate at 80° C. for 10 minutes, thereby obtaining one coating film of photosensitive resin composition (1) and one coating film of comparative photosensitive resin composition (1). Each of the coating films was exposed to light in a predetermined pattern. Thereafter, the coating film of photosensitive resin composition (1) and that of comparative photosensitive resin composition (1) were heated at 155° C. for 10 minutes each.

### <Evaluation of Photosensitive Resin Compositions>

### (Pattern Forming Ability)

The coating film produced by using photosensitive resin composition (1) and exposed to light at 500 mJ/cm² in a predetermined pattern, was heated on a hot plate at 140° C. for 10 minutes. Then, it was immersed in a mixed solution of a 2.38 wt % tetramethylammonium hydroxide aqueous solution and isopropanol at 9:1. As a result, a pattern in which an exposed region was not dissolved in the developer and remained, was obtained. In addition, the patterned coating film was heated at 350° C. for one hour for imidization. It is clear from this result that the photosensitive resin composition of the present invention can form an excellent pattern.

On the other hand, comparative photosensitive resin composition (1) finally formed a pattern at 2,000 mJ/cm² after conducting an experiment in the same manner as that of photosensitive resin composition (1), except that the heating temperature after the exposure was 155° C.

### Examples 2 to 9

### Production of Photosensitive Resin Compositions (2) to (9)

Photosensitive resin compositions (2) to (9) were produced in the same manner as photosensitive resin composition (1), except that base generators (2) to (9) were used in place of base generator (1).

### (Production of Coating Film)

Each of photosensitive resin compositions (2) to (9) was spin-coated on a chrome-plated glass plate so as to have a final film thickness of 4 μm and dried on a hot plate at 80° C. for 10 minutes, thereby obtaining coating films of photosensitive resin compositions (2) to (9) (one coating film for each photosensitive resin composition). Each of the coating films was exposed to light in a predetermined pattern. Thereafter, the coating films of photosensitive resin compositions (2) to (9) were heated at 150° C. for 10 minutes each.

### (Pattern Forming Ability)

Pattern formation was performed by using each of photosensitive resin compositions (2) to (9) in the same manner and thus each of them succeeded at 500 mJ/cm². It is clear from this result that the photosensitive resin composition of the present invention can form an excellent pattern.

### Example 10

### Production of Photosensitive Resin Composition (10)

Photosensitive resin composition (10) having the following composition was produced by using base generator (1) of the present invention.

Epoxy resin: (YP50EK35 (phenoxy resin), 35 wt % methyl ethyl ketone solution (manufactured by Nippon Steel Chemical Co., Ltd.): 100 parts by weight

Base generator: 10 parts by weight

Photosensitive resin composition (10) was spin-coated on a glass plate so as to have a final thickness of 0.5 μm and dried on a hot plate at 80° C. for 15 minutes, thereby obtaining two coating films. One of the two coating films was subjected to whole surface exposure at 100 J/cm² by means of a manual exposure device and a high pressure mercury lamp. Then, each of the coating films was heated at 150° C. for 60 minutes. The heated coating films were immersed in a mixed solution of isopropanol and chloroform (isopropanol:chloroform=4:1 (by volume ratio)) at room temperature for 10 minutes. As a result, it was found that the coating film exposed and then heated was not dissolved in the mixed solution, and the epoxy resin was cured in the film. On the other hand, the other coating film heated but not exposed was dissolved in the mixed solution.

### Example 11

### Production of Photosensitive Resin Composition (11)

Photosensitive resin composition (11) was produced, comprising hexamethylene diisocyanate (manufactured by Kanto Chemical Co., Inc.) of 100 parts by weight as an isocyanato resin, polytetrahydrofuran (manufactured by Aldrich Corp.) of 150 parts by weight as a resin having a hydroxyl group, base generator (1) of 10 parts by weight and tetrahydrofuran of 500 parts by weight.

Photosensitive resin composition (11) was spin-coated on a chrome-plated glass plate so as to have a final film thickness of 0.5 μm and dried on a hot plate at 60° C. for five minutes, thereby obtaining one coating film of the photosensitive resin composition. The thus-obtained coating film was subjected to whole surface exposure at 100 J/cm² by means of a manual exposure device and a high pressure mercury lamp. Then, the coating film was heated at 120° C. for 10 minutes and cooled to room temperature. As a result, a low-elastic solid was obtained, and it was confirmed that curing of the isocyanato and hydroxyl groups was promoted.

### Example 12

### Production of Photosensitive Resin Composition (12)

Photosensitive resin composition (12) was produced by mixing alkoxysilane condensate (1) obtained in Synthesis example 2 of 100 parts by weight with base generator (1) of 10 parts by weight and then dissolving the mixture in tetrahydrofuran of 500 parts by weight, which is a solvent.

Photosensitive resin composition (12) was spin-coated on two chrome-plated glass plates so as to have a final film thickness of 0.5 μm and dried on a hot plate at 80° C. for five minutes, thereby obtaining two coating films of the photosensitive resin composition. One of the coating films of the photosensitive resin composition was subjected to whole surface exposure at 100 J/cm² by means of a manual exposure device and a high pressure mercury lamp. Then, each of the exposed and unexposed coating films was heated at 120° C. for 30 minutes. Infrared absorption spectral measurement was performed on each of the samples before and after the heating. As a result, in the exposed and heated coating film sample, a peak at 1,020 cm⁻¹ appeared, which is assigned to an Si-β—Si bond that indicates occurrence of polymerization, and peaks at 2850 cm⁻¹ and 850 cm⁻¹ decreased, which are assigned to Si—OCH₃ that indicates raw materials, compared to those of the same before the heating. In the unexposed and heated coating film sample, a peak at 1,020 cm⁻¹ also appeared, which is assigned to an Si—O—Si bond that indicates occurrence of polymerization; however, the peak was smaller than the exposed coating film. From these results, it is clear that when exposed to light, the base generator of the present invention generates a base and thus promotes polymerization of the alkoxysilane condensate.

### Example 13

### Production of Photosensitive Resin Composition (13)

Photosensitive resin composition (13) having the following composition was produced.

Base generator (8): 100 parts by weight

Solvent (tetrahydrofuran or THF): 300 parts by weight

### (Production of Coating Film)

Photosensitive resin composition (13) was spin-coated on a chrome-plated glass plate to have a final film thickness of 4 μm and dried on a hot plate at 80° C. for 10 minutes, thereby obtaining one coating film of photosensitive resin composition (13). The thus-obtained coating film was exposed to light at 1,000 mJ/cm² in a predetermined pattern by means of a high pressure mercury lamp. Thereafter, the coating film was heated at 160° C. for 10 minutes and then immersed in a mixed solution of a 2.38 wt % tetramethylammonium hydroxide aqueous solution and isopropanol at 9:1. As a result, a pattern in which an exposed region was not dissolved in the developer and remained, was obtained.

1. A base generator which comprises a compound having two or more partial structures each represented by the following general formula (1) per molecule and generates a base by exposure to electromagnetic radiation and heating: 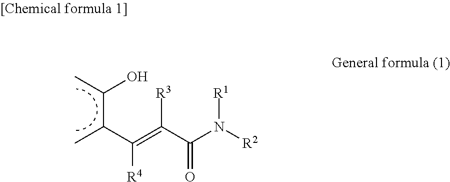
wherein R¹ and R² are each independently a hydrogen or an organic group and may be the same or different; at least one of R¹ and R² is an organic group; R¹ and R² may be bound to form a cyclic structure which may contain a heteroatom but does not contain an amide bond; R³ and R⁴ are each independently one selected from the group consisting of a hydrogen, a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group and an organic group and may be the same or different.

2. The base generator according to claim 1, which is a compound represented by the following general formula (2), a compound having a repeating unit represented by the following general formula (2′) or a compound represented by the following general formula (3): 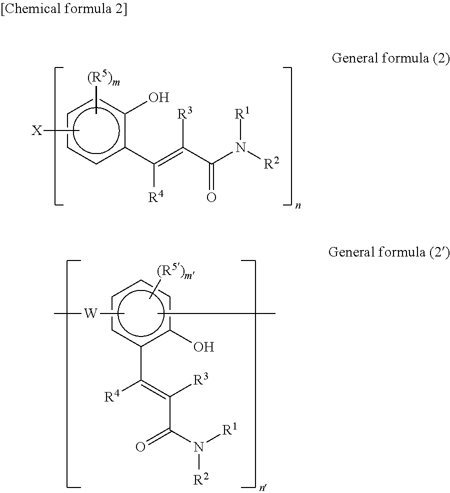
wherein R¹, R², R³ and R⁴ are the same as those of the general formula (1); n or n′ R¹s may be the same or different; n or n′ R²s may be the same or different; n or n′ R³s may be the same or different; n or n′ R⁴s may be the same or different; X is a direct bond or n-valent chemical structure to which two or n structures shown in the brackets are bound; W is a direct bond or a divalent linking group; n and n′ are each an integer of 2 or more; R⁵ and R^{5′} are each independently one selected from the group consisting of a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group, an amino group, an ammonio group and an organic group; m is 0 or an integer of 1 to 3; m′ is 0 or an integer of 1 or 2; two or more R⁵s may be the same or different and may be bound to form a cyclic structure which may contain a heteroatom; and two or more R^{5′}s may be the same or different and may be bound to form a cyclic structure which may contain a heteroatom; 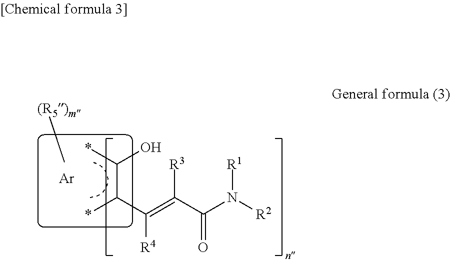
wherein R¹, R², R³ and R⁴ are the same as those of the general formula (1); n″ R¹s may be the same or different; n″ R²s may be the same or different; n″ R³s may be the same or different; n″ R⁴s may be the same or different; Ar is an aromatic hydrocarbon which has 6 to 24 carbon atoms and which may have a substituent, and which has n″ partial structures shown in the brackets; n″ is an integer of 2 or more; R^{5″} is one selected from the group consisting of a halogen, a hydroxyl group, a mercapto group, a sulfide group, a silyl group, a silanol group, a nitro group, a nitroso group, a sulfino group, a sulfo group, a sulfonato group, a phosphino group, a phosphinyl group, a phosphono group, a phosphonato group, an amino group, an ammonio group and an organic group; m″ is 0 or an integer of 1 or more; and two or more R^{5″}s may be the same or different and may be bound to form a cyclic structure which may contain a heteroatom.

3. The base generator according to claim 1, having a 5% weight loss temperature of 100° C. or more and 350° C. or less.

4. The base generator according to claim 1, having absorption at least one of electromagnetic wavelengths of 365 nm, 405 nm and 436 nm.

5. A photosensitive resin composition comprising a polymer precursor in which reaction into a final product is promoted by a basic substance or by heating in the presence of a basic substance, and any one of the base generators defined by claim 1.

6. The photosensitive resin composition according to claim 5, wherein the polymer precursor comprises one or more kinds selected from the group consisting of a compound having an epoxy group, isocyanate group, oxetane group or thiirane group, a polymer having an epoxy group, isocyanate group, oxetane group or thiirane group, a polysiloxane precursor, a polyimide precursor and a polybenzoxazole precursor.

7. The photosensitive resin composition according to claim 5, wherein the polymer precursor is soluble in basic solutions.

8. The photosensitive resin composition according to claim 5, wherein the polymer precursor is a polyimide precursor or polybenzoxazole precursor.

9. A photosensitive resin composition comprising a polymer having a repeating unit represented by the following general formula (2-4) as an essential component: 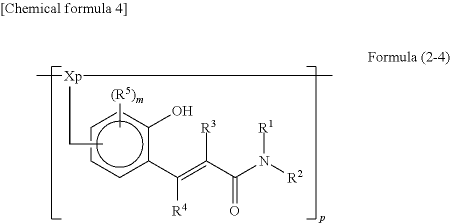
wherein R¹, R², R³, R⁴, R⁵ and m are the same as those of the general formula (2); Xp is a repeating unit of the polymer; and p is a number of 2 or more.

10. The photosensitive resin composition according to claim 5, which is usable as a paint, a printing ink, a sealing agent or an adhesive, or as a material for forming display devices, semiconductor devices, electronic components, microelectromechanical systems, stereolithography products, optical elements or building materials.

11. A pattern forming material comprising any one of the photosensitive resin compositions defined by claim 5.

12. A pattern forming method by forming a coating film or molded body with any one of the photosensitive resin compositions defined by claim 5, exposing the coating film or molded body to electromagnetic radiation in a predetermined pattern, heating the coating film or molded body after or at the same time as the exposure to change the solubility of the exposed region, and then developing the coating film or molded body.

13. An article selected from a printed product, a paint, a sealing agent, an adhesive, a display device, a semiconductor device, an electronic component, a microelectromechanical system, a stereolithography product, an optical element or a building material, wherein at least part of each of which articles comprises any one of the photosensitive resin compositions defined by claim 5 or a cured product thereof.

14. The photosensitive resin composition according to claim 9, which is usable as a paint, a printing ink, a sealing agent or an adhesive, or as a material for forming display devices, semiconductor devices, electronic components, microelectromechanical systems, stereolithography products, optical elements or building materials.

15. A pattern forming material comprising any one of the photosensitive resin compositions defined by claim 9.

16. A pattern forming method by forming a coating film or molded body with any one of the photosensitive resin compositions defined by claim 9, exposing the coating film or molded body to electromagnetic radiation in a predetermined pattern, heating the coating film or molded body after or at the same time as the exposure to change the solubility of the exposed region, and then developing the coating film or molded body.

17. An article selected from a printed product, a paint, a sealing agent, an adhesive, a display device, a semiconductor device, an electronic component, a microelectromechanical system, a stereolithography product, an optical element or a building material, wherein at least part of each of which articles comprises any one of the photosensitive resin compositions defined by claim 9 or a cured product thereof.
